(19) United States
(12) Patent Application Publication
(10) Pub. No.: US 2007/0250270 A1
(43) Pub. Date: Oct. 25, 2007

(54) Cellular predictive models for toxicities

(75) Inventors: Eugeni A. Vaisberg, Foster City, CA (US); Vadim Kutsyy, Los Altos, CA (US)

Correspondence Address: BEYER WEAVER LLP, P.O. BOX 70250, OAKLAND, CA, 94612-0250, US

(73) Assignee: Cytokinetics, Inc.

(21) Appl. No.: 11/715,664

(22) Filed: Mar. 7, 2007

Related U.S. Application Data
(60) Provisional application No. 60/781,489, filed on Mar. 9, 2006.
(60) Provisional application No. 60/782,707, filed on Mar. 14, 2006.

(30) Foreign Application Priority Data
Mar. 17, 2006 (GB) 0605357.3

(51) Int. Cl. G01N 33/48

(52) U.S. Cl. 702/19

(57) ABSTRACT

Methods for generating models for predicting biological activity of a stimulus test population of cells are provided. The models may be used to classify or predict the effect of stimuli on cells. In certain embodiments, the methods involve receiving data comprising values for dependent variables associated with stimuli; preparing a set of cell populations based on the data received; identifying a subset of the cell populations to be used in generating a model from data associated with the subset, wherein the model is provided to predict activity of a test population.

### CROSS-REFERENCE TO RELATED PATENT APPLICATIONS

This application claims priority under 35 U.S.C. § 119(e) to U.S. provisional applications Nos. 60/781,489 filed on Mar. 9, 2006 and 60/782,707 filed on Mar. 14, 2006, both titled CELLULAR PREDICTIVE MODELS FOR TOXICITIES, hereby incorporated by reference for all purposes. This application also claims priority under 35 U.S.C. § 119 to Great Britain application No. 0605357.3, filed Mar. 17, 2006 and also titled CELLULAR PREDICTIVE MODELS FOR TOXICITIES, hereby incorporated by reference for all purposes.

This application is related to the following patent applications: U.S. application Ser. No. 10/623,486 (Patent Publication No. 20050014216), filed Jul. 18, 2003 and titled PREDICTING HEPATOTOXICITY USING CELL BASED ASSAYS; U.S. application Ser. No. 10/719,988 (Patent Publication No. 20050014217), filed Nov. 20, 2003, also titled PREDICTING HEPATOTOXICITY USING CELL BASED ASSAYS; U.S. application Ser. No. 11/651,885, filed Jan. 9, 2007 and titled DOMAIN SEGMENTATION AND ANALYSIS; U.S. application Ser. No. 11/651,912, filed Jan. 9, 2007 and titled GRANULARITY ANALYSIS IN CELLULAR PHENOTYPES; U.S. application Ser. No. 11/653,109, filed Jan. 12, 2007 and titled RANDOM FOREST MODELING OF CELLULAR PHENOTYPES; and U.S. application Ser. No. 11/653,096, filed Jan. 12, 2007 and titled ASSAY FOR PHOSPHOLIPIDOSIS.

This application is also related to the following concurrently filed patent applications: U.S. patent application Ser. No. ______ (Atty Docket No. CYTOP164) titled CELLULAR PREDICTIVE MODELS FOR TOXICITIES; U.S. patent application Ser. No. ______ (Atty Docket No. CYTOP165), also titled CELLULAR PREDICTIVE MODELS FOR TOXICITIES; U.S. patent application Ser. No. ______ (Atty Docket No. CYTOP166), also titled CELLULAR PREDICTIVE MODELS FOR TOXICITIES; and U.S. patent application Ser. No. ______ (Atty Docket No. CYTOP167), titled NORMALIZING CELL ASSAY DATA FOR MODELS. These applications are incorporated herein by reference for all purposes.

Methods of building and applying models to predict toxicities based on phenotypic characteristics are provided. In certain embodiments, methods of modeling the effects of stimuli on cellular populations using appropriate training sets are provided. In certain embodiments, a random forest algorithm is employed to generate decision tree models.

In drug discovery, valuable information can be obtained by understanding how a potential therapeutic affects a cell population. Insight may be gained exposing a compound to a stimulus (e.g., a genetic manipulation, exposure to a compound, radiation, or a field, deprivation of required substance, or other perturbation). The ability to quickly determine whether a population of cells exhibits a particular pathology or other classification provides a valuable tool in assessing the mechanism of action or toxicity of an uncharacterized stimulus that has been tested on the population of cells

Models of various forms may be used to classify and/or predict behavior of populations of cells using a large number of previously classified cell populations. It would desirable to have additional models that are able to accurately predict or classify effects of diverse array of stimuli on the cell populations.

Some aspects of modeling disclosed herein pertain to generating models for classifying stimuli based on hepatotoxicity. Such models may be characterized by the following operations: (a) receiving images of hepatocytes which have been exposed to stimuli and treated with one or more markers for cellular components in the hepatocytes; (b) extracting two or more phenotypic features from the one or more markers in the images; (c) providing a training set comprising data points including data about the phenotypic features and hepatotoxicity; and (d) from the training set, generating a model classifying stimuli according to whether they are hepatotoxic. In some embodiments, the data points comprise (i) the two or more phenotypic features and (ii) an indication of the presence or absence of hepatotoxicity in the stimuli applied to the hepatocytes from which the phenotypic features were obtained. The features may be automatically extracted from particular regions of the image, which regions may have been identified by segmentation. In some cases, the features are derived from whole cell regions occupied by hepatocytes. In other cases, they are derived from particular regions within hepatocytes such as nuclei, peripheral regions of cells, granules within the cells, etc. Certain embodiments, employ features extracted from regions corresponding to granules and/or peripheral regions within the hepatocytes

Other disclosed methods pertain to computer-implemented methods for classifying a stimulus according to whether it is hepatotoxic. Such methods may be characterized by the following operations: (a) receiving at least one image of hepatocytes which have been exposed to the stimulus and treated with one or more markers for cellular components in the hepatocytes; (b) automatically extracting two or more phenotypic features from the one or more markers in the image (c) applying the two or more phenotypic features to a model that classifies stimuli according to whether they are hepatotoxic; and (d) receiving a hepatotoxicity classification for the stimulus as an output from the model. As with the method of building model just described, the features used in this method may be extracted from various regions of the image identified by segmentation. In some cases, the features are taken from hepatocytes on a whole cell basis. In other cases, they are derived from particular regions within hepatocytes such as nuclei, peripheral regions of cells, granules within the cells, combinations of these, etc.

In certain embodiments, methods for generating models to classify or predict the hepatotoxic effect of stimuli on cells involve (a) receiving a data set comprising values for dependent variables associated with stimuli; (b) preparing a set of cell populations treated with said stimuli; (c) identifying a subset of the treated cell populations to be used in generating a model for classifying hepatotoxicity of stimuli; and (d) generating said model from phenotypic data associated with the subset. The model may be employed to classify stimuli based on hepatotoxic effects they produce in a test population of cells. Methods of classifying hepatotoxicity of a stimulus may involve applying phenotypic data associated with cells treated with the stimulus to a model.

Still other embodiments described herein pertain to computer-implemented methods of classifying a cell or population of cells by pathology or toxic response. These methods may be characterized by the following operations: (a) receiving a set of phenotypic features of the cell or population of cells; (b) in a multi-dimensional phenotypic feature space, calculating a measure of difference (e.g., a distance) between at least a first subset of the set of phenotypic features of the cell or population of cells and corresponding phenotypic features of a negative control; (c) determining that the measure of difference calculated in (b) is greater than a threshold value; (d) providing a second subset of the set of phenotypic features from the cell or population of cells as an input to a model for classifying cells based on pathology or toxic response; and (e) receiving a pathology or toxic response classification for the cell or population of cells as an output from the model. Certain embodiments make a determination of whether data from cells or populations of cells have a measure of difference that is greater than the threshold value. Only the data for these “active” cells or populations is applied to the classification model.

Additionally, certain methods of producing a model for classifying cells according to a pathology or toxic response may be characterized as follows: (a) receiving data points, each comprising (i) a set of phenotypic features of a cell or population of cells and (ii) an indication of whether the pathology or toxic response is present; (b) in a multi-dimensional phenotypic feature space, calculating a measure of difference for each of the data points, between at least a first subset of the set of phenotypic features of the data point and corresponding phenotypic features of a negative control; (c) identifying those data points having a measure of difference as calculated in (b) that is greater than a threshold value; and (d) applying an algorithm to the data points identified in (c) to thereby create a model for classifying cells according to the pathology or toxic response based on a second subset of the set of phenotypic features.

Various implementations of the above methods are provided. For example, the first subset of phenotypic features and the second subset of phenotypic features may be different or identical. Further, the measures of difference may be calculated as a Euclidean distance or a Manhattan distance. In addition, the model for classifying cells based on pathology or toxic response may comprise a decision tree. In certain embodiments, all phenotypic features may be obtained automatically by image analysis.

The above methods may be employed to assess a pathology or toxic response associated with hepatocytes. In such cases, the cell or population of cells may comprise a hepatocyte or population of hepatocytes. In some cases, the pathology classification is one or more of cholestasis, steatosis, and phospholipidosis.

Note that certain embodiments do not rely on difference methods; i.e., they do not employ a measure of difference between phenotypic features of test data and corresponding phenotypic features of a negative control. For example, certain embodiments employ all data from all cell populations (wells) regardless of their “activity” (calculated phenotypic difference from a negative control) to build a classification model. Such models may be used in a manner such that data from any well (regardless of activity level) exposed to a stimulus is submitted for classification.

At the other extreme, certain approaches build classification models by employing only data from active wells. In such embodiments, model building involves first identifying active wells from among all wells (both positive and negative, for example known cholestatic and non-cholestatic stimuli), and using only active wells to build a model. When using such models, only data from active wells is submitted for classification.

Other approaches involve intermediate applications of data from active wells. In one example, models may be built using training sets comprising active wells for a positive class (e.g., cholestatic stimuli) together with data from all wells that for the negative class (e.g., non-cholestatic stimuli) regardless of level of activity in the wells of the negative class. When using such models, data from any well (regardless of activity level) exposed to a stimulus may be submitted to a model for classification. In this approach, there is no need to identify active wells prior to submitting the resulting data to the model for classification.

In another example, model building employs a training set comprised of three classes: active wells from a positive class (e.g., cholestatic compounds), active wells from a negative class (e.g., non-cholestatic compounds), and all wells from a negative control (e.g., wells treated with DMSO). The resulting models may classify stimuli (or cells or populations of cells) according to these three classes. Applying such models to classify cells or stimuli may involve submitting data from any well (regardless of activity level) exposed to a stimulus. As with the immediately prior approach, there is no need to identify active wells prior to submitting the resulting data to the model for classification. A classification from the model indicating that a cell or stimulus is in the same class as the negative control would effectively indicate an inactive stimulus.

Further aspects of the invention pertain to computer-implemented methods that involve normalization of phenotypic data as part of the process for classifying a stimulus as to toxicity or pathology. Similarly, aspects of the invention pertain to methods that involve normalization of phenotypic data in the process of producing models for classifying a stimulus as to toxicity or pathology.

Certain embodiments involve (a) obtaining one or more phenotypic features from one or more images of cells exposed to a stimulus or stimuli, and (b) normalizing the phenotypic features obtained in (a) using corresponding phenotypic features extracted from one or more images of cells in a negative control. In the context of classifying stimuli, the methods may further involve (c) applying the normalized phenotypic features to a model for classifying stimuli as to toxicity or a pathology associated with cells, and (d) receiving a classification of the stimulus from the model. In the context of producing a model, the methods may involve providing a training set comprising data points, and generating a model from the training set. Each data point may include (i) the one or more phenotypic features, as normalized in (b) above, and (ii) an indication of the presence or absence of the toxicity or pathology caused by the stimuli applied to the cells from which the phenotypic features were obtained.

In some cases, the normalizing operation ((b) above) comprises subtracting mean values of the phenotypic features of the cells of the negative control from values of the phenotypic features of the cells exposed to the stimulus or stimuli, to thereby provide feature difference values. Normalizing may further involve dividing the feature difference values by standard deviations of the corresponding phenotypic features from the cells of the negative control. In such cases, the corresponding phenotypic features from the negative control may be obtained from multiple negative control wells, which may be provided on one or multiple different plates. In certain embodiments, the mean values of the corresponding phenotypic features from the cells of the negative control are obtained from multiple negative control wells on a single plate. The single plate may include wells for both the cells of the negative control and the cells exposed to the stimulus. As explained elsewhere, the cells of the negative control may be treated with DSMO.

The features, markers, stimuli, and models may any of those described elsewhere herein. Thus, generally the phenotypic features may comprise at least one of (i) intensities of a marker within cell populations and (ii) morphologies of a marker within cell populations. Further, the phenotypic features may be obtained from one or more segmented regions within the cell images. Examples of the segmented regions include granules, nuclei, and peripheral regions within the cells, as well as the whole cells themselves. Further, the model may assume the form of a decision tree or an ensemble of decision trees.

In certain embodiments, the cell lines to which the model applies are hepatocytes and the pertinent model classifies stimuli as to hepatotoxicity or a pathology associated with hepatocytes (e.g., one or more of cholestasis, steatosis, and phospholipidosis).

In addition to the above-described methods, the invention pertains to computer program products including machine-readable media on which are stored program instructions for implementing various models. Any of the methods described herein (in whole or part) may be represented, in whole or in part, as program instructions that can be provided on such computer readable media.

These and other features and advantages of the above embodiments will be described in more detail below, with reference to the associate drawings as appropriate.

### BRIEF DESCRIPTION OF THE DRAWINGS

FIG. 1 is a flowchart depicting one method of producing a model that can be used to classify or predict activity a population of cells.

FIG. 2 is a simple example of a data set to be used in building a model.

FIG. 3 is a flowchart one method of identifying active wells.

FIG. 4 is a flowchart depicting one method for determining whether a particular stimulus and level of stimulus is active.

FIG. 5 is a flowchart depicting one method for building a random tree model.

FIG. 6A is a schematic illustrating a rough example of a partially-grown random tree model.

FIG. 6B is a schematic illustrating variable selection for a node of a random tree model.

FIG. 7 is a flowchart depicting a high level method for evaluating data using a model.

FIG. 8 is a schematic block diagram of an image capture and image processing system that can be used in accordance with certain embodiments described herein.

Methods for building and applying models to predict the effects of stimuli on cell populations are provided. In certain embodiments, the models predict whether a stimulus will induce particular pathologies or other activities. Such models may classify a stimulus as positive or negative for a particular pathology.

In certain embodiments, the methods for building a model employ a training data set containing independent variables associated with cell populations (e.g., phenotypic characteristics such as intensity and morphological features of markers located within the cells) and at least one dependent variable that classifies the cell populations according to pathology and/or toxicity. Examples of pathology classifications include cholestatis or steatosis. An example of a toxicity classification is hepatotoxicity. In accordance with certain embodiments, the independent variables include cellular phenotype features obtained by automated image analysis.

In certain embodiments, dependent variables employed in a training data set are obtained from information showing the effects of certain stimuli on cells; e.g., the toxicity of various chemical compounds. Such information may be available in the literature, from private sources, by internal research, etc. The information may be in vivo data associated with the stimuli. The independent variables employed in the training data set may be obtained by exposing cell populations to the stimuli. In some cases, the stimuli are employed at multiple levels such as multiple concentrations of a chemical compound. In each case, phenotypic features of the treated cells are extracted and used in conjunction with the associated dependent variables to produce the data set.

In certain embodiments, the training data set is used with an appropriate model generation algorithm such as (i) a random forest technique to produce decision trees, (ii) a regression technique such as partial least squares, or (iii) a technique for generating neural networks. The resulting models may be employed to predict or classify the toxicity of known compounds on a particular cell type.

As indicated, training data sets may be generated using data from cell populations treated with particular stimuli. The term “cell population” is used interchangeably with “population of cells.” A population of cells may include one or more cells. In certain embodiments, a population of cells is the cells in a well on a plate. For purposes of discussion, the term “wells” may be used to reference any region occupied by cell populations. In certain embodiments, a population of cells is the cells in a field of view used in obtaining an image of cells in a well or other support medium.

As indicated, models of this invention may be used to assess the impact of particular stimuli applied to the cell populations. Many types of stimuli are appropriate and include organic and inorganic materials such as biomolecules, small molecules, etc., pathogens, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include temperature, pressure, acoustic energy, electromagnetic radiation, the lack of a particular material (e.g., the lack of oxygen as in ischemia), temporal factors, etc. Various levels of stimuli may be applied to cell populations. For purposes of discussion, reference is primarily made to compounds at concentrations. However, the discussion extends to other stimuli.

As indicated, in certain embodiments, the models use cellular phenotypic features as “independent variables” or “inputs” when using a model. Numerous cellular phenotypic features, also referred to as descriptors, are known to be useful in predicting a condition or classifying a stimulus. Some of these are described in the following patent documents, each of which is incorporated herein for all purposes: U.S. Pat. No. 6,876,760 titled CLASSIFYING CELLS BASED ON INFORMATION CONTAINED IN CELL IMAGES, U.S. Patent Publication No. 20020144520 titled CHARACTERIZING BIOLOGICAL STIMULI BY RESPONSE CURVES, U.S. Patent Publication No. 20020141631 titled IMAGE ANALYSIS OF THE GOLGI COMPLEX, U.S. Pat. No. 6,956,961 titled EXTRACTING SHAPE INFORMATION CONTAINED IN CELL IMAGES, U.S. Patent Publication No. 20050014131 titled METHODS AND APPARATUS FOR INVESTIGATING SIDE EFFECTS, U.S. Patent Publication No. 20050009032 titled METHODS AND APPARATUS FOR CHARACTERISING CELLS AND TREATMENTS, U.S. Patent Publication No. 20050014216 titled PREDICTING HEPATOTOXICITY USING CELL BASED ASSAYS, and U.S. Patent Publication No. 20050014217, also titled PREDICTING HEPATOTOXICITY USING CELL BASED ASSAYS, U.S. Provisional Patent Application No. 60/509,040, filed Jul. 18, 2003 and titled CHARACTERIZING BIOLOGICAL STIMULI BY RESPONSE CURVES, U.S. patent application Ser. No. 11/098,020, filed Apr. 1, 2005 and titled METHOD OF CHARACTERIZING CELL SHAPE, U.S. patent application Ser. No. 11/155,934, filed Jun. 16, 2005 and titled CELLULAR PHENOTYPE, U.S. patent application Ser. No. 11/192,306, filed Jul. 27, 2005 and titled CELL RESPONSE ASSAY EMPLOYING TIME-LAPSE IMAGING and U.S. patent application Ser. No. 11/082,241, filed Mar. 15, 2005 and titled ASSAY FOR DISTINGUISHING LIVE AND DEAD CELLS.

General categories of features include marker intensity and morphological characteristics. These features are typically determined on a per cell basis and then averaged or aggregated over the multiple cells in an image. Typically, though not necessarily, the phenotypic characterizations are derived in whole or in part by automated image analysis.

Intensity values correlate to marker concentration. High marker concentrations at particular locations correspond to high signal intensities at pixels associated with the particular locations. Examples of intensity related features include location, population size, and various statistical values. The statistical features typically pertain to a concentration or intensity distribution or histogram. Specific examples include mean, standard deviation or variance, skewness, and kurtosis of intensity values within a defined region. The defined region within which such intensity values are evaluated may include, for example, the boundary of a cell, an organelle (e.g., a nucleus), one or more granules, a peripheral region of a cell, etc. Examples of morphological features include various shape and size characteristics such eccentricity, axis ratio for an object fit to an ellipse, perimeter, area, etc.

Some specific examples of feature types suitable for use with this invention include various whole cell and nucleus features where appropriate, cell or object counts, an area, a perimeter, a length, a breadth, a fiber length, a fiber breadth, a shape factor, an elliptical form factor, an inner radius, an outer radius, a mean radius, an equivalent radius, an equivalent sphere volume, an equivalent prolate volume, an equivalent oblate volume, an equivalent sphere surface area, a mean intensity, a total intensity, an optical density, a radial dispersion, and a texture difference. These features can be mean or standard deviation values, or frequency statistics from the parameters collected across a population of cells. Further examples employing specific markers will be presented below for models of predicting hepatotoxicity. The phenotypic characterizations may also be derived in whole or in part by techniques other than image analysis.

Various markers may be considered in developing models for classifying stimuli by effect; e.g., hepatotoxicity and associated pathologies. Among the markers that have been found to provide features useful for modeling hepatotoxicity are markers for cytoskeletal proteins and structures, canaliculae and proteins therein, endocytic machinery, Golgi components, mitochondria, nuclei, general protein content within a cell, and lipids. In some cases, such markers are employed to show the biological states relevant to hepatotoxicity such as ploidy states.

Generally, a marker provides a signal that is captured on an image showing the location of the marker with respect to a cell or particular cellular components. In other words, the location of the signal source (i.e., the location of the marker within the cells) appears in the image. To this end, the marker may be luminescent, radioactive, fluorescent, etc. The labeling agent typically emits a signal at an intensity related to the concentration of the cell component to which the agent is linked. For example, the signal intensity may be directly proportional to the concentration of the underlying cell component.

Various stains and compounds may serve as markers. As examples, markers may be designed to bind to particular components already existing a cell (e.g., fluorescently labeled antibodies to particular proteins), be expressed as part of cellular protein (e.g., fusion proteins including yellow fluorescent protein), be transported through a cell (e.g., a labeled tubulin or a labeled phospholipid), etc. Specific examples of such compounds include fluorescently labeled antibodies to the cellular component of interest, fluorescent intercalators, and fluorescent lectins. The antibodies may be fluorescently labeled either directly or indirectly. A few examples of markers relevant to toxicity such as hepatotoxicity will now be briefly described.

Cytoskeletal markers attach to particular cytoskeletal proteins and/or assemblies thereof such as actin, tubulin, microtubules, actin filaments, etc. Examples of tubulin markers include fluorescently labeled antibodies to tubulin (e.g., DM1-α, YL1-2, and 3A2 antibodies), labeled tubulin, and the like. Various hepatocyte pathologies including steatosis and cholestasis have an impact on cytoskeletal proteins.

Markers to canalicular structures and tight junctions may be used in some models of hepatotoxicity and associated pathologies. These include markers for various proteins typically found in canaliculae such as actin, BSEP, and MRP2. As explained elsewhere herein, a change in the canaliculae may indicate cholestasis or other form of hepatocyte pathology such as steatosis.

Other markers relevant to hepatotoxicity include markers to endocytic structures such as the Golgi apparatus. Examples of markers include antibody markers to the TGN protein p-38 as well as labeled lens culinaris lectin (LC lectin) or antibodies to proteins enriched in the Golgi complex, such as gp130, [beta]COP. The TGN is responsible for transporting BSEP, which has been implicated in hepatotoxicity, particularly cholestasis.

Mitochondrial markers such as markers for cytochrome C have been found useful in certain models for hepatotoxicity. Some pathologies cause release of cytochrome C from mitochondria followed by migration of the cytochrome C into other regions of the cell. Hence features characterizing the morphology and/or intensity of cytochrome C may be employed in models of this invention. In certain embodiments, Green Fluorescent Protein (GFP) and/or antibodies also can be used to identify the presence of cytochrome C outside the mitochondria. See, e.g., Goldstein et al. (2000) Nature Cell Biol. 2:156; and Ogawa et al. (2002) Intl. J. Molecular Medicine 10:263. Changes of mitochondrial membrane potential have also been implicated in hepatotoxicity.

As discussed elsewhere herein, nuclear markers may be employed to segment images to identify cells as well as identify particular features having specific relevance to hepatotoxicity models. DNA markers include fluorescently labeled antibodies to DNA and fluorescent DNA intercalators such DAPI and Hoechst 33341 available from Invitrogen Corporation of Carlsbad, Calif. The nuclei may also be imaged using histone markers such as GFP-histone2B fusion protein, antibodies to the phosphorylated histones such as (pH3). Note that during mitosis, the histones in the nucleus become phosphorylated. Therefore, mitotic index is measured using a pH3 marker will also give a high reading for the stimuli that induce mitotic arrest.

Other reagents for segmenting cells include non-specific markers for proteins. Examples include succinimidyl esters conjugated to fluorescent dyes such as TAMRA or Alexa-Fluor dyes. These reagents label primary amine groups of proteins and can be useful in identifying cells within an image. They may also be used to distinguish live and dead cells. The Alexa 647 nm succinimidyl ester reagent (A647SE available from Invitrogen Corporation of Carlsbad, Calif.) may be used to segment individual hepatocytes within an image.

Lipid markers may bind to neutral or phospholipids. Examples of markers that bind to neutral lipids are Bodipy and Nile Red. In some embodiments described herein, labeled DHPE is employed to mark the transport of phospholipids within hepatocytes. Lipid transport and accumulation is important in at least steatosis and phospholipidosis.

Those of skill in the art will understand that the methods of producing and using models as described herein may be applied to cells and biological materials other than hepatocytes. Toxicity in other cells such as myocytes, neurons, etc. may be considered in the same manner by generating and using models according to the description herein.

In certain embodiments, producing models for classifying stimuli on the basis of pathology or other biological effect involves first identifying particular stimuli and/or associated levels (e.g., concentrations) of such stimuli that produce a reasonably strong effect on cells and then using information from only the strongly effecting stimuli/level as a training set for producing a decision tree or any other kind model. Various techniques may be employed to determine whether a level of a particular stimulus has a sufficiently strong effect on cells. In some embodiments, these techniques involve determining phenotypic differences between cells treated with the stimulus and cells in a negative control. One approach involves determining a measure of difference between phenotypic features of the treated and control cells within a multi-dimensional phenotype space. As an example, a Euclidean or Manhattan distance may be calculated in the multidimensional feature space. Regardless of how one determines whether a particular stimulus or level of stimulus is “active,” the training set for building a model may be limited, in some embodiments, to data from active wells or cell populations. Likewise, in certain embodiments, classification of stimuli using models may be limited to those stimuli or levels of stimuli found to be active. Other embodiments, which are described below, employ all stimuli or levels of stimuli regardless of whether they are found to be active.

Most of the discussion in this application pertains to generation and use of models in the form of decision trees. The invention is not limited in this manner. Any form of model may be employed. Examples include, in addition to decision trees, mixture models, linear expressions, non-linear expressions, neural networks, support vector machines, classification algorithms based on distances or differences in multi-dimensional phenotypic space, etc.

In some embodiments, the model takes the form of a decision tree or a group of decision trees. As described below, appropriate decision tree models may be produced using a random forest technique. In certain embodiments, the random forest technique (or other suitable technique) may be employed to produce an ensemble of decision tree models, which are used together to classify cells and the stimuli applied to them. The separate decision trees of the ensemble may be produced using multiple bootstrap samples. In certain embodiments, the bootstrap samples are produced using clustering and/or stratification constraints. As explained below, clustering may be performed based on particular stimuli, with each cluster being a collection of phenotypic data points for various levels of the stimuli (e.g., concentrations of a particular compound applied to a cell population). The bootstrap samples maybe stratified based on the proportions of various pathologies (or other biological effects) in the original data set.

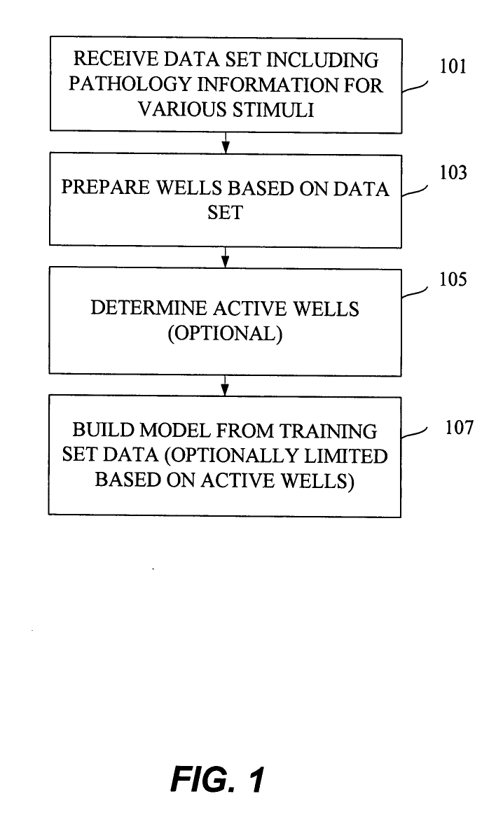

FIG. 1 shows a high-level flow chart illustrating steps in building a model to predict biological activity according to certain embodiments of the present invention using information about cell populations and/or stimuli. In an operation 101, data including information about one or more pathologies associated with particular stimuli is received. The information may be a binary (yes/no) or graded prediction that indicates whether or not the cell population exhibits certain pathologies or other biological effects (e.g., whether a particular stimulus applied to the cell population induces a particular pathology or other effect). If the model is to be used to determine whether a cell or population of cells exhibits a particular pathology, then the pathology information in the data set may serve as a dependent variable. Although the example shown in FIG. 1 refers to pathology information, the data set may include information about biological conditions in addition to or instead of pathology information (e.g., whether a potential therapeutic stimulus is likely to have a particular side effect, its mechanism of action, etc.).

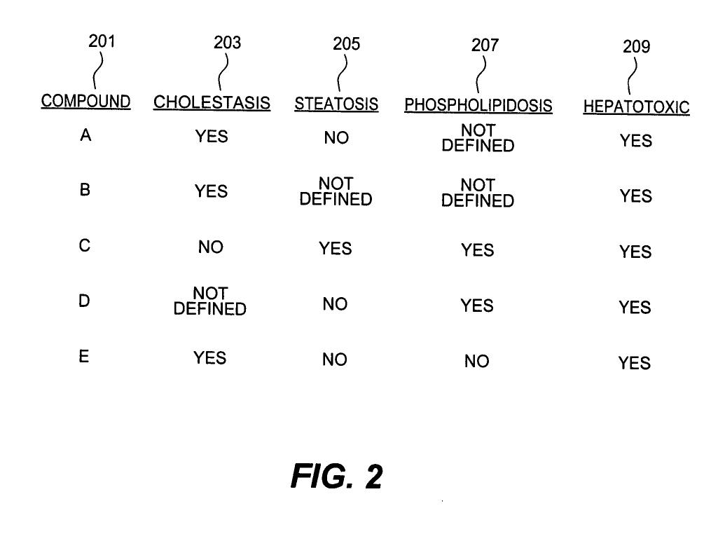

A simple example of such data received in operation 101 is presented in FIG. 2. The column indicated by reference number 201 identifies different compounds, and the columns indicated by reference numbers 203-209 identify particular pathologies (the dependent variables) that might result from treatment with the compounds. The values in columns 203-207 indicate whether or not the compounds induce the associated pathologies in a cell type or types under consideration. In the example presented in FIG. 2, three pathologies that may be exhibited by hepatocytes are shown, specifically cholestasis, steatosis and phospholipidosis.

The example in FIG. 2 shows a binary (yes/no) classification for each pathology. In certain embodiments, a predictive score may be used in place of the binary classification. The score may indicate how strongly the toxicity or pathology is exhibited in cells treated with the stimulus (e.g., a percent or degree of activity). Some data may also be provided with a confidence value indicating a level of confidence that the compound or other stimulus induces the named pathology may be provided in some embodiments. Such information may be employed to “weight” or discard particular data in generating a model. In some cases, the effectiveness of a compound in inducing a pathology may be unknown or not defined; in such cases, the compounds are given the annotation “not defined.”

The invention is not limited to models for classifying stimuli on the basis of toxicity or induced pathology. Examples of other dependent variables (classifications) include whether a stimulus induces mitotic arrest, whether it produces “off-target” effects (potential side effects), etc. Examples of non-binary classifications that provide state-based classifications include where in the cell cycle a particular cell currently resides, the mechanism of action of a particular stimulus such as a compound, etc.

In the example shown in FIG. 2, reference number 209 indicates whether the compound is considered overall hepatotoxic. Hepatoxicity describes compounds that induce any one or more of the listed pathologies (steatosis, cholestasis, and phospholipidosis) and/or other conditions of hepatocytes such as necrosis, carcinoma, is a PPAR (peroxisome proliferators-activator receptor), etc.

The individual data points in the data set shown in FIG. 2 are identified by a stimulus, in this case a compound. This information coupled with experimentally derived phenotypic features is then used to build a model to predict biological activity of cells. While the data points depicted in this example are tied to particular identified stimuli, this need not be the case. In some embodiments, training set data points are comprised of only a dependent variable (e.g., whether a particular condition or effect is exhibited in cells) and independent variables (e.g., specific phenotypic features characterizing the cells).

In some embodiments, the models are built using cell populations treated with compounds at multiple concentrations. Certain phenotypic features characteristic of cell populations treated with the compounds (at the multiple concentrations) serve as the inputs or independent variables in the model. A first cell population may be identified as being treated with compound a at concentration c₁, a second cell population identified as being treated with compound a at concentration c₂, etc. A compound may induce a pathology at all concentrations, only at certain concentrations, or not at all. In certain embodiments, the data received in operation 101 may indicate whether the compound induces a pathology at a particular concentration; in other embodiments, the data may indicate only whether the compound induces a pathology without any indication of the concentrations at which it induces the pathology. In certain embodiments involving the latter case, models may be built using only data from cell populations treated with concentrations of a compound sufficient to induce a significant response in the cell populations.

After the stimulus-condition data is received in operation 101, phenotypic features induced by the particular stimuli may be collected from individual cell populations or wells, each exposed to a unique stimulus (e.g., a particular compound at a particular concentration). See operation 103. As indicated, cell populations may include one or more cells. The stimuli applied to the cell populations or wells prepared in operation 103 are chosen based on the data received in operation 101.

One or more wells may be treated with a discrete combination of stimulus and level of stimulus in order to produce a data point used as the stimulus/level data point used in generating the model. In this approach, a data point is comprised of (1) information about a pathology or other biological effect (e.g., whether the associated stimulus is known to induce cholestasis), (2) phenotypic features (and possibly other features) derived from a population of cells treated with the stimulus at a defined level), and optionally (3) the identity of the stimulus and its level of application. For example, for the data set shown in FIG. 2, wells may be prepared by treating a first well with compound a at concentration c₁, treating a second well with compound a at concentration C₂, etc. until each compound is represented by 10 different concentrations associated with 10 different wells. In certain embodiments, replicate wells may be prepared (i.e., multiple wells having the same compound at the same concentration, or more generally the same stimulus applied at the same level). Also, as discussed below, in some embodiments multiple plates containing identical or matched wells may be prepared for performing multiple assays. In any case, the data (particularly phenotypic data) taken from these wells is employed to build a predictive model of pathology or other biological effect.

After the pathology information or other dependent variables associated with cell populations is provided, “active” wells (or other cell populations) to be used in building the model are optionally determined in operation 105. Active wells are wells in which the stimulus applied induces some reasonable effect on the cells. Data from wells for which the applied stimulus has little or no effect on cells is not, in certain embodiments, used in building the model. In this approach, only some of the available data points (information derived from wells treated with a particular stimulus) available to generate the model are selected for use in building the predictive model. Those wells (associated with particular stimuli/levels of stimuli) deemed or determined to be “active” are used to build the model. Data from other wells (“inactive” wells) are not employed to build the model. In other embodiments, data from all wells, active and inactive, is used to build the model.

In embodiments where the data received in operation 101 includes concentration-dependent information about the effect of compounds, operation 105 may involve only selecting those concentrations at which compounds are classified as having some effect on the cells (or at which a predictive score or confidence value is above a certain threshold). Concentrations at which compounds are not believed to induce the effect to be modeled are deemed “inactive” and not, in such embodiments, included in the data set employed to build the model. Hence data need not be generated from compounds at these concentrations.

In other embodiments, including embodiments for which the data set received in operation 101 is not annotated with concentration information, detecting active wells may involve comparing each well with a reference point (e.g., a negative control) to determine if the compound/concentration applied to the well has a substantial or reasonable effect on the biological activity of the cells.

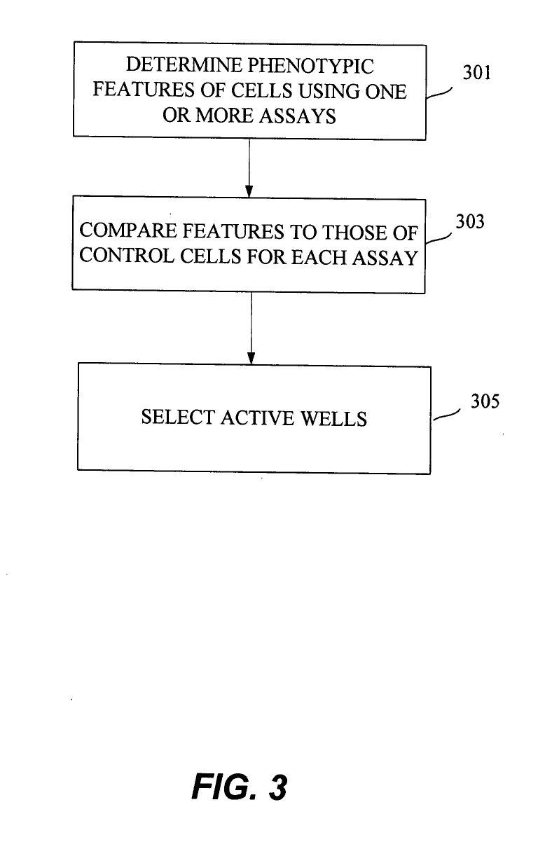

FIG. 3 shows a flow chart depicting operations of a method of determining active wells according to certain embodiments. In an operation 301, one or more assays are run to determine various phenotypic features of the cell populations in the wells. As indicated, in certain embodiments, features are obtained by analyzing a cell image showing the positions and concentrations of one or more markers associated with particular cellular components (e.g., DNA, Golgi, particular receptors, particular cytoskeletal proteins, etc.). At every combination of compound, dose, and optionally cell line and staining protocol, one or more images can be obtained. As explained, these images are used to extract various parameter values of relevant cellular features.

Generally a given image of a cell population, as represented by one or more markers, can be analyzed in isolation or combination with other images of the same cell population, as represented by different markers, to obtain any number of image features. As explained above, most features may be characterized as either marker intensity measures or morphological characteristics. Intensity values correlate to marker concentration. High marker concentrations at particular locations correspond to high signal intensities at pixels associated with the particular locations. Examples of intensity related features include location, population size, and various statistical values. The statistical features typically pertain to a concentration or intensity distribution or histogram. Specific examples include mean, standard deviation or variance, skewness, and kurtosis of intensity values within a defined region. The defined region within which such intensity values are evaluated may include, for example, the boundary of a cell, an organelle (e.g., a nucleus), one or more granules, a peripheral region, etc. Examples of morphological features include various shape and size characteristics such eccentricity, axis ratio for an object fit to an ellipse, perimeter, area, etc.

The phenotypic features associated with a particular stimulus/level of stimulus may be obtained from one well or multiple wells and/or from one or multiple images. Each well provides data on a discrete population of cells treated with a particular stimulus at a particular level. Multiple assays, each typically using different markers and generating a different collection of features, may be run on multiple plates each containing identical or matched wells (e.g., wells with identical cell lines treated with identical compounds/concentration). Also in some embodiments, replicate wells are used—for example, compound a may be used to treat three cell populations at each concentration. Thus, for example, if each assay has 10 different concentrations of compound a and 3 replicates, the compound is represented by 30 points in multi-dimensional space (10 concentrations times 3 replicates).

Once the phenotypic features are obtained for each well, all or a subset of features may be compared to control wells to measure the effect of the compound/concentration on the cells in operation 303. In this manner, “active” wells (data points) may be identified and selected for model building. In the case of experiments based on application of compounds, the control wells may be produced by treating cells in a well with stimulus that is essentially inert (i.e., has little or no biological effect). In certain embodiments, control wells are wells on the same plate treated with DMSO (dimethyl sulfoxide). Of course, various other methods of measuring the effect of the compound/concentrations may be used, including comparing the phenotypic features to a different normalization point.

A general method of determining whether a particular stimulus and associated level of that stimulus is sufficiently active for use in building a model involves determining whether cellular phenotypic data associated with that level of stimulus is sufficiently different from phenotypic data associated with a negative control (e.g., DMSO treated cells). The phenotypic difference may be measured by various techniques including a distance in phenotype space, a multi-dimensional Kolmogorov-Smirnov or T² test, an inverse split regression technique, etc. Most of the discussion hereafter assumes a distance in phenotype space is employed to identify active stimuli. Note that the phenotypic features employed to determine such distance need not be the same features employed in models ultimately generated from the data.

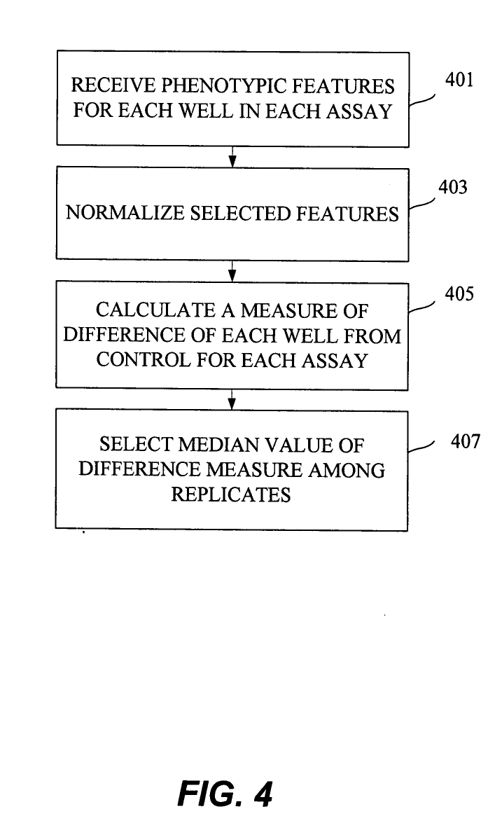

In accordance with certain embodiments, a method for determining whether a particular stimulus and level of stimulus is “active” is depicted in the flow chart of FIG. 4. These embodiments assume multiple replicates are prepared for each stimulus/level of stimulus combination. They also assume that the phenotypic data used to calculate phenotypic distance is derived from multiple different assays. Finally, these embodiments also assume that calculating a separation between phenotypic features from test wells and phenotypic features from DMSO-treated wells (or other negative control wells) involves calculating the Euclidean distance in multi-dimensional space. Note that other measures of distance may be employed such as a Manhattan distance. The multi-dimensional space comprises phenotypic features as dimensions; e.g., mean DNA marker intensity is one dimension, average cell area is a second dimension, etc.

In this method as illustrated in FIG. 4, the phenotypic features for each assay are received in an operation 401 (i.e., the features measured in operation 301). Most or all dimensions (biological features) of each well are scaled or normalized to a comparable range of values in an operation 403. In one example, this is accomplished by subtracting the mean value of a particular biological feature of the DMSO wells from a particular plate from each of the features of the wells on that plate. (Because the imaging conditions may vary from plate to plate, only the mean of the DMSO values from the particular plate of the well in question are subtracted.) Each feature may be further scaled by dividing the scaled values by one or both of the standard deviation of DMSO values for the feature as measured across multiple plates and the standard deviation of all values for the feature as measured across multiple plates. The scaling of any value of a particular biological feature (dimension) in operation 403 may be given by the following expression:

$${X}_{normalized} = \frac{X - {μ}_{DMSO on the plate}}{{σ}_{DMSO across multiple plates}}$$

where X is an unscaled value of the feature for a particular well, μ_{DMSO on the plate} is the is the mean value of the biological feature across the DMSO values on the plate and σ_{DMSO across multiple plates} is the standard deviation of the feature values of the DMSO wells across multiple plates. Multiple plates indicates that the standard deviation is calculated from available data values, and not the only the values on the particular plate. The available data values may come from multiple plates in an experiment or from historical data. As indicated, σ_{DMSO across multiple plates} may be replaced by the standard deviation of the values across all wells of the multiple plates or each feature may be scaled by dividing by both quantities.

After normalization of the variables, the Euclidean distance or any other measure of difference such as Manhattan L1 distance of each well from the DMSO controls is calculated in an operation 405 for each assay. The Euclidean distance is the square root of the sum of the squares of each of the dimensions (features) and the distance d(X) for each well may be calculated by

$$d ( X ) = \sqrt{{\sum }_{i = 1}^{n} {( {X}_{i} - {\overline{X} ( control )}_{i} )}^{2}}$$

where the mean X(control)ᵢ terms are zero if the features have been centered on DMSO (or other control) in operation 403. The median value of the replicates distances is taken in an operation 407 to eliminate outlying data.

Returning to FIG. 3, once the wells have been compared to a normalization point in operation 303, active wells or concentrations are selected in an operation 305. For example, once the distances for each well are calculated, active wells may be determined by selecting those wells that have median distances greater than a threshold distance. It should be noted that in the example shown in FIG. 4, a distance is calculated for each of multiple assays. For example, if there are five assays each having a different threshold distance, five different distances are calculated for each compound/concentration. Note that each assay may employ the same set of stimulus/level wells but measure features from different markers. A well (e.g., a unique compound/concentration combination) may be designated as active if any of the five distances exceed the threshold. In alternate embodiments, data from all assays may be combined to generate a single distance within a large multi-dimensional space comprised of dimensions taken from all assays. This single distance is compared against a threshold to determine if the stimulus/level is active. In another approach, a stimulus/level is deemed active only if all or some of the multiple distances (one for each assay) are greater than specified thresholds. Examples of assays and the features used to calculate distances according to a specific embodiment are shown below.

The processes shown in FIGS. 3 and 4 are examples of a method that may be used to determine active wells. Other methods may be used as well. Further discussions of normalizing dimensions, distance calculations and measuring effects of stimuli may be found in the following applications, which are hereby incorporated by reference for all purposes, U.S. Patent Publication No. 20020155420 titled CHARACTERIZING BIOLOGICAL STIMULI BY RESPONSE CURVES and U.S. Patent Publication No. 20050137806 titled CHARACTERIZING BIOLOGICAL STIMULI BY RESPONSE CURVES. Determining active wells may also be accomplished by other suitable methods including, as mentioned, inverse split regression, multi-dimensional Kolmogorov-Smirnoff, T² methods, random forest and other methods. Manually inspecting each image by eye is another method for detecting active wells. Also in certain embodiments, a concentration may be classified as active if at least a certain percentage (e.g., 25%) of cells are dead. See, e.g., the above-referenced patent application U.S. patent application Ser. No. 11/082,241, titled ASSAY FOR DISTINGUISHING LIVE AND DEAD CELLS (published as U.S. Patent Publication No. 20060014135) and U.S. patent application Ser. No. 11/355,258, filed Feb. 14, 2006 and titled ASSAY FOR DISTINGUISHING LIVE AND DEAD CELLS (published as U.S. Patent Publication 20070031818), hereby incorporated by reference in their entireties. Any of these methods may be used alone or in combination with one another (e.g., a well is active if any of the distances meets a threshold or if more than 25% of cells are dead.).

Referring again to FIG. 1, determining active wells in operation 105 results in a training set of compound/concentrations that have a reasonable effect on the cell lines of interest, each of which is annotated with a binary classification or other predictive value for the pathology (or other dependent variable) of interest. It should be noted that for a particular pathology, only the compounds that are annotated as positive or negative may be used in the model. In addition, the training set contains independent variable values (including phenotypic feature values extracted from cell populations treated with the particular compound concentrations) that will be used in building the model. In embodiments where multiple assays are used to obtain feature values, there may be duplicate feature values (e.g., two or more assays may calculate cell area). In these cases, only one of the feature values (e.g., randomly selected from amongst the assays or mean value of the feature taken across multiple assays) may be used in building the model to not give these features undue importance.

The above discussion assumes that a training set includes employs only data from “active” stimuli for building models of hepatotoxicity. In such embodiments, any stimuli, even those known to be hepatotoxic at certain levels, are not used in the training set if they do not elicit a response found to be active.

The overall process may be summarized as follows. Cells treated with a stimulus under investigation are first analyzed to determine whether they are “active.” As indicated, this may involve a determination of whether the treated cells sufficiently different in a phenotypic sense from negative control cells (completely inactive cells). If the treated cells are not found to be active, they are deemed non-hepatotoxic and their features are not applied to the model. If, however, the treated cells are found to be active, their relevant phenotypic features are applied to the model, which classifies the stimulus on the basis of hepatotoxicity.

In other embodiments, the training set for building the model draws on data from additional sources. In one case, the training set includes data from not only the “active” stimuli but from negative controls and non-hepatotoxic stimuli as well. In such embodiments, all data is used in the training set except for data from inactive hepatotoxic stimuli. As an example, a low concentration of a known hepatotoxic compound produces cells whose phenotypic changes are insufficient to be deemed active. Data from such treatment would not be included in the training set. Models produced using this combination of data from active and inactive stimuli would, in certain embodiments, be used to directly classify any stimulus under investigation, regardless of whether it would be first characterized as active. Hence, an initial step of determining activity is not required with such models. Note that in these embodiments, the concepts of hepatotoxic and non-hepatotoxic stimuli can be generalized to positive and negative classes of stimuli. Thus, if for example a model is designed to classify stimuli for some specific pathology such as cholestasis, the positive and negative classes might include cholestatic and non-cholestatic compounds. In such example, building the model would include cholestatic stimuli as one class, and non-cholestatic stimuli as another.

In a third example, the training set for the hepatotoxicity model includes data from all active stimuli as well as data from a negative control (e.g., hepatocytes treated with DMSO). In this example, the training set will not include data from any inactive stimuli (regardless of whether the stimuli is known to be hepatotoxic or not) except for the negative control data. Again, the concept can be generalized from hepatotoxicity models to models for particular pathologies.

As with other aspects of the invention described herein, it should be understood that the three above examples may be applied to cells and biological materials other than hepatocytes. Toxicity in other cells such as myocytes, neurons, etc. may be considered in the same manner by generating and using models according to the above guidelines. The training sets may be selected using any combination of active and inactive wells from stimuli known to be toxic and non-toxic as described above for hepatocytes.

Models are built using the training set data in operation 107. As indicated, the training set data is optionally limited to active wells—at least for some classes of training set data. Decision tree models are one form of model that may be employed in this invention. In certain embodiments, methods provided herein use bootstrapping techniques. Bootstrapping methods involve generating bootstrap samples from an original data set. These bootstrap samples may then be used to generate models of various forms, with decision trees being one example. Bootstrap samples are created by sampling, with replacement, from an original data set to create a new data set (a bootstrap sample) of the same or different size as the original data set. In the methods provided herein, the bootstrap samples are used to generate random forest models. Bootstrap methods have been shown to improve the robustness of tree models and allow additional analysis of the model (such as variable selection and estimation of the future performance of the model).

In conventional bootstrap techniques, the bootstrap sample is selected by sampling, with replacement, individual data points from the original data set. In certain embodiments of methods provided herein, however, the data set is clustered prior to generating the bootstrap samples. Clustering involves grouping cell populations by a parameter or characteristic. In certain embodiments, the cell populations are clustered by stimulus, for example by compound. Thus, all cell populations treated with compound a will be in cluster a, all cell populations treated with compound b will be in cluster b, etc. The bootstrap samples are built by randomly sampling clusters, with replacement, to build a sample of the size of the original data set (in terms of number of clusters) or another predetermined sample size. For example, if the original data set contains 100 members, and each cluster has 10 members, building each bootstrap sample involves selecting 10 clusters from the clustered data set. Each cluster may be of different size.

As indicated above, in certain embodiments, the data set is stratified in addition to clustered. The bootstrap samples are then built by randomly sampling clusters, with replacement, within each stratum. In this manner, each bootstrap sample has the same proportion of clusters belonging a particular stratum as the original data set. For example, if there are 400 compounds known to induce cholestasis, 100 compounds that do not induce cholestasis, the data may be divided into strata, the first stratum containing 400 compounds and the second containing 100. The data set may then be clustered within each stratum prior to bootstrap sampling.

In addition to pathology, the data set may also be stratified by other parameters, such as chemical properties. Also in certain embodiments, the data set may be sub-stratified. For example, cell populations not exhibiting cholestasis may be further stratified by another pathology or chemical properties, such as exhibiting or not exhibiting steatosis, being part of chemical series or other parameters. Also as indicated above, in cases in which stratification is performed, the bootstrap samples are built by random sampling of clusters within each strata. In this manner, the ratio of the sizes of the strata is maintained. For example, if the data set is stratified by pathology, each bootstrap sample will contain the same proportion of positive (pathology inducing) to negative compounds as the original data set.

Because the bootstrap samples are built by random sampling of clusters, the likelihood that a particular compound will not be represented in a bootstrap sample (and corresponding random forest model) is greatly increased and equal to 1/e˜=32.7%. For example, if a training set contained 100 wells treated with 10 different compounds, a random sampling of individual wells, with replacement, would almost surely have representatives of each compound. Bootstrap samples generated according the methods of the present, however, are far likelier not to contain any wells treated with a particular compound. This is important because the resulting models are more robust, that is they are able to accurately predict classifications for cells treated with a diverse array of compounds in the future data (or predict classifications for a diverse array of whatever parameter is used to cluster).

In certain embodiments, the decision tree models are random forest models. U.S. patent application Ser. No. 11/653,109, filed Jan. 12, 2007 titled RANDOM FOREST MODELING OF CELLULAR PHENOTYPES, which is hereby incorporated by reference, discusses random forest modeling of cellular phenotypes. Random forest algorithms use bootstrap samples to generate individual decision trees. The trees are grown by selecting a random subsample of the independent variables at each node and selecting the variable that produces the best outcome.

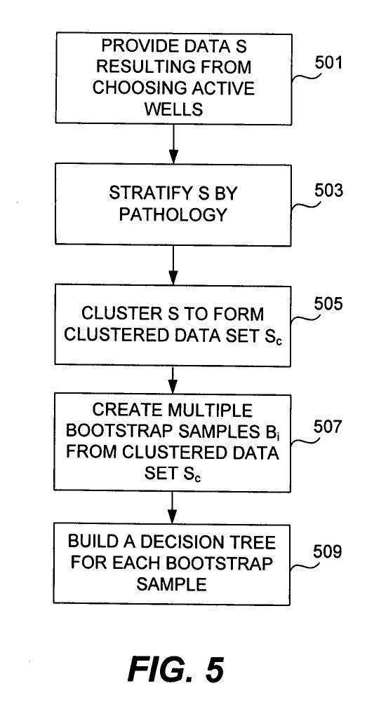

One method of generating a random forest model is shown in FIG. 5. The method begins at operation 501 where an original data set S having data about m cell populations is provided. In the example shown in FIG. 5, S is the data set resulting from choosing active wells. The data set may also be referred to as a training set and includes biological classifications/predictions and phenotypic features (i.e., the dependent and independent variables values) for all cell populations across all compounds, concentrations, replicates, cell lines, etc. For example, each data point in the set may correspond to a population of cells in a well treated with a certain compound at a certain concentration and the independent and dependent variables associated for that well. In operation 503, the data set S is stratified by pathology. For example, in building a model for classifying cells as exhibiting cholestasis or not, the data set may stratified by dividing the data set into populations treated with compounds that are known to induce cholestasis (at any concentration) and those that do not. Thus, if compounds a and b are annotated as cholestasis compounds but compounds c and d are not, the population corresponding to compounds a and b put into the first stratum, and the population corresponding to compounds c and d are put into the second stratum. In operation 505, the data set S is clustered to form a clustered data set S_{c}. Clustering the data set involves grouping data points based on a shared parameter. For example, if data points are clustered by compound, all data points corresponding to compound a are put in cluster a, all data points corresponding to compound b are put in cluster b, etc.

From the clustered data set S_{c}, multiple bootstrap samples Bᵢ are created in operation 507. Each of these is obtained by sampling, with replacement, from the clustered data set to create a new set with m members. The “with replacement” condition produces variations on the original set S. A bootstrap sample, Bᵢ, will sometimes contain replicate samples from S and lack certain samples originally contained in S. Also, because the data set is clustered, selecting a cluster insures all data points in that cluster will be contained in the bootstrap sample Bᵢ. It should be noted that when the data set is stratified, each bootstrap sample is obtained by sampling, with replacement, from each stratum such that the ratio of the sizes of the strata (in terms of number of clusters) is the same as in the original data set.

At operation 509, an unpruned decision tree is built for each bootstrap sample Bᵢ in accordance with the random forest algorithm. At each node of the tree, a subset of independent variables are randomly sampled and tested to determine how well it predicts the dependent variable at the current node. The variable providing the best result is then taken from this subset. In this manner, an unpruned tree is grown for each bootstrap sample Bᵢ. The ensemble of all the trees makes up a model that may be applied to data to predict or classify the pathology or activity.

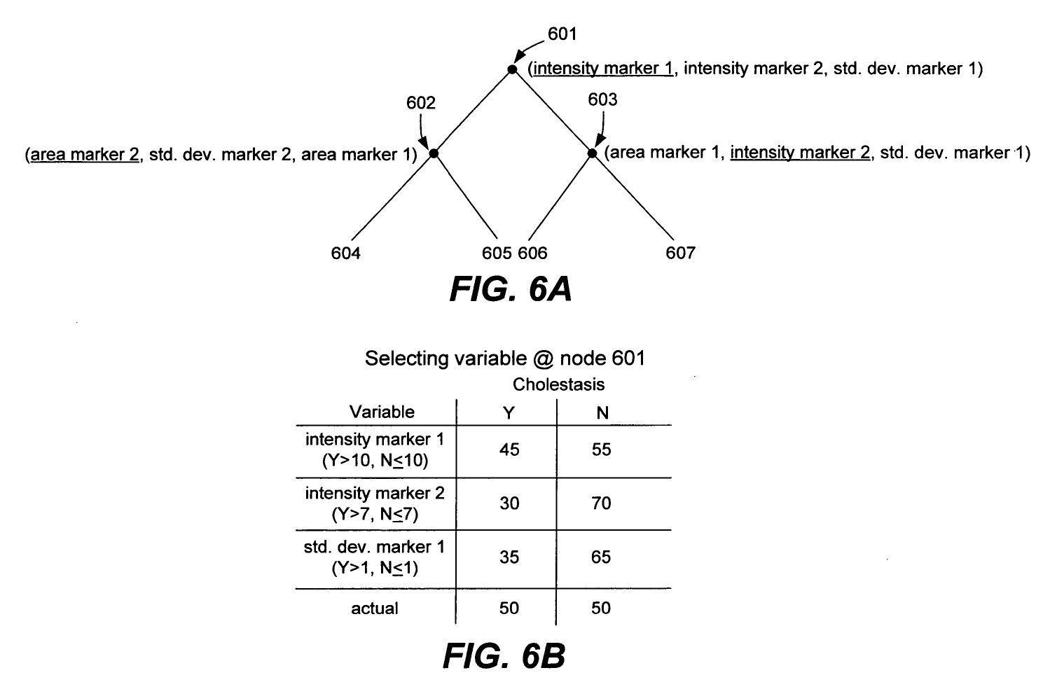

A simple example of building a random forest model is illustrated in FIGS. 6A and 6B. In this example, there are 6 independent variables associated with each well in the bootstrap sample: the intensity of marker 1, the intensity of marker 2, the standard deviation of the intensity of marker 1, the standard deviation of the intensity of marker 1, the standard deviation of the intensity of marker 2, the area of marker 1 and the area of marker 2. The bootstrap sample contains the values of these independent variables for all wells. The bootstrap sample also contains the values of the dependent variable, in this example whether the cells in the well exhibit cholestasis or not. In this example, the size n of the random subset of independent variables is 3. Thus, 3 of the variables are randomly selected for the first node, in FIG. 6A, node 601. In this example, intensity of marker 1, intensity of marker 2, and standard deviation of marker 1 are the variables randomly selected for node 601. Each of these variables is then tested to find the one that best predicts the known outcomes. FIG. 6B shows results of testing each of the randomly selected variables. Applying decision criteria for the first variable, the intensity of marker 1 (Y if >10, N if ≦10), to the bootstrap sample predicts that cells in 45 wells exhibits cholestasis and 55 do not. Decision criteria for the other selected variables is applied as well. As can be seen in FIG. 6B, the prediction made by basing the decision on intensity of marker 1 is closest to the actual results; thus this variable is chosen as the variable on which to base the decision at node 601 in the model. This is indicated in FIG. 6A by the line under the selected variable. Other cost functions such as the Gini index may be also used for tree building. The tree is then grown, producing two more nodes, nodes 602 and 603. The process of randomly selecting a subset of variables and selecting the best variable on which to base decision is repeated for these nodes. The data is filtered through the previous nodes prior to selecting the best variable; for example selecting the best variable at node 602 is based only on the 45 wells that were predicted “Y” at node 601. The tree is grown, producing nodes 604-607 as shown. Steps 605-607 are repeated to grow the tree. The tree is considered complete or grown when each of the nodes contains only a single class, i.e. a prediction of 100%.

FIGS. 6A and 6B illustrate generating a decision tree for a single bootstrap sample. Referring back to FIG. 5, operation 509, a decision tree or random tree model is grown for each of the bootstrap samples. The ensemble of these trees (i.e., the forest) may be then be used to classify cell populations based on the values of the independent variables associated with them. The example shown in these figures results in a binary (Y/N) classification. As indicated above, the random forest algorithm may also be used to build regression trees that return numerical values. For example, a number from 0 to 1 may be used to indicate the likelihood of a cell population exhibiting the activity. Building regression trees employs different cost functions, such as sum of squares of errors, but the process is otherwise similar to building classification trees in that it also includes a single value prediction for each of the nodes.

Random forest algorithms provide information about the relative importance of the independent variables in predictions. (See, e.g., Leo Breiman, “Random Forests—Random Features,” Technical Report 567, University of California, Berkeley, September 1999 and Svetnik et al. “Random Forest for Classification and Regression in QSAR Modeling”, which are hereby incorporated by reference). In certain embodiments, after building the model as described above (e.g., using features from the multiple assays), the model may be rebuilt using only features that are determined to have a certain level of importance. If the results are comparable, the model using the smaller number of independent variables is used. This process may be repeated one or more times to find smaller subsets of independent variables that provide results comparable to the initially built model. A similar process may also be used to identify the relative importance of multiple assays.

Further discussion of the building random forest models may be found in above-referenced U.S. patent application Ser. No. 11/653,109.

As indicated above, the random forest models may be built using all wells for which there is a known classification or prediction for the pathology or other activity of interest. In an alternate embodiment, the models may be built using DMSO cell populations as well. For example, a random forest model for cholestasis may be built using three types of wells: active wells that are positive for cholestasis, active wells that are negative for cholestasis, and DMSO wells. The model may then used to classify the test populations into one of three classifications: positive, negative or DMSO-like. Alternatively, a model may classify test populations into positive or negative/DMSO-like.

FIGS. 5 and 6 describe methods of building random forest models according to certain embodiments. One of skill in the art will understand that various modifications may be made to the described process. Other types of models may also be used including, for example, logistic models for PLD.

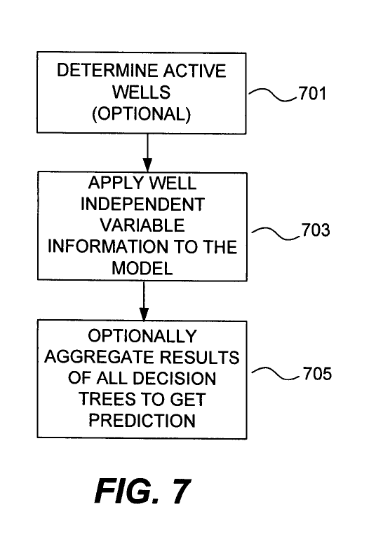

FIGS. 1-6 describe processes of building classification and regression models for pathologies according to certain embodiments. The models may then be used to classify or predict biological activity of test cell populations (e.g., a population of cell treated with a test compound or other stimulus suspected of inducing a pathology). FIG. 7 is a simple flowchart illustrating three high level steps in applying a model to classify a stimulus or its effect on a test population of cells according to certain embodiments. The process begins at an operation 701 in which active wells are optionally determined as discussed above. In one example, multiple concentrations of a particular compound may be used to treat wells. Only active concentrations or concentrations at which compounds have a reasonable effect on the cell population (e.g., as determined by a comparison to control wells), are applied to any or all of the models. If none of the concentrations are deemed active, then the compound may be deemed inactive. Methods described above for determining active concentrations (e.g., determining whether a Euclidean distance is greater than a particular threshold) may be employed for this purpose.

In some embodiments, as indicated above, data from any well, regardless of level of activity, is provided to the model. In such cases operation 701 is not performed. Rather the independent phenotypic data is provided directly the classification model in use and a stimulus is classified directly.

Independent variables (e.g., phenotypic features) taken from wells (active wells in some embodiments) are applied to the model in an operation 703. This operation involves applying the independent variables for each well to the model, which is a collection of random forest trees in the example presented here. The independent variables are the same as those used to generate the model as described above, and in certain embodiments, describe phenotypic characteristics of the population. The independent variables are typically obtained by performing the same assays as used to build the model.

Unlike the data provided in the training set, the dependent variable (e.g., does the cell exhibit cholestasis or not) is not known for the population of cells—this is what the model determines. The data is applied to each tree in the ensemble of trees generated as discussed above with regard to FIGS. 5 and 6. Each tree produces a result or prediction. In certain embodiments, the prediction is binary (yes/no) indicating that the population of cells exhibit or do not exhibit the pathology or classification of interest. In certain embodiments, the result is a numeral indicator of the pathology or classification.

As explained above, some methods for building models will produce models that do not require an initial step of filtering stimuli for activity. To use such models, one can apply the phenotypic features to the model directly. Such models may provide a negative control (e.g., treatment with a DMSO-like compound or other control) as a one potential output (dependent variable), in addition to activity for the pathology in question and activity but not for the pathology in question. In evaluating raw data with such models, operation 701 (identifying active wells) may be avoided as the model includes a DMSO-like classification or prediction.

In an operation 705, the predictions of all the trees are aggregated. In certain embodiments, the predictions are aggregated by majority vote (e.g., for binary classification). In certain embodiments, the predictions are aggregated by averaging (e.g., for numerical predictions). The aggregate of the predictions of the trees is the result or prediction for the test population or concentration. For example, in certain embodiments, each cell population (e.g., each compound/concentration used to treat the populations) receives a prediction from 0-1 that indicates the likelihood that the cell population exhibits the pathology.

The well-based prediction or classification information may be analyzed in various ways to give compound-based information (or information on other types of stimulus). In embodiments where replicate wells are used, the median prediction value may be used to eliminate outlier data. In certain embodiments, all concentrations that have predictions of at least a threshold prediction value may be identified as positive for the pathology (i.e., inducing the pathology). A minimum concentration at which an effect is evident may also be identified using the threshold. In certain embodiments, the maximum prediction over all concentrations of a compound may be used as an overall prediction of the pathology-inducing ability of the compound.

### ASSAY EXAMPLES

As discussed above, assays are used in certain embodiments to generate the phenotypic features employed to build, apply models, or both. In certain embodiments, assays include subjecting cells to one or more stimuli, imaging the cells, and analyzing one or more cell images showing the positions and concentrations of one or more markers located within the cells. A given assay may be characterized by the collection of markers or features employed to define a cellular phenotype. The features obtained are typically chosen to have some relationship to the biological activity or effect of interest. The following examples of assays obtain features likely to be related to hepatotoxicity, in some cases including one or more hepatotoxic pathologies. Examples of features obtained by the assays are also listed; in one example, the listed features are used to measure separation of phenotypic features obtained in test wells from phenotypic features obtained in on or more control wells, e.g., wells in which the cells are treated with DMSO.

As indicated above, typical features obtained by the assays may be roughly divided into morphological features and intensity-based features. Morphological features include features that describe, e.g., size, area and elongation (e.g., by axis ratios) and are not specific to a particular marker. Intensity-based features are marker specific and include features that describe total and mean intensities as well as other statistical properties of the intensity of a marker such skewness and kurtosis, which may indicate if the material labeled by the marker (e.g., protein, DNA, etc.) is punctate or smooth, for example. Some intensity-based features also relate to texture.

Intensity-based features also include features associated with granularity. Granularity refers to bright spots or granules typically found within a cell or some subcellular region. In some cases, granules found by image analysis represent intercellular organelles or other objects in images. Phenotypic features associated with granularity include number of granules, area of granules and intensity of granules. Extracting features associated with granularity from an image is described in U.S. patent application Ser. No. 11/651,912, filed Jan. 9, 2007, titled GRANULARITY ANALYSIS IN CELLULAR PHENOTYPES, which is hereby incorporated by reference for all purposes.

In certain embodiments, obtaining feature values may first involve identifying the locations of the discrete cells in the image. This may be accomplished by segmentation. Segmentation can be performed by various techniques including those that rely on identification of discrete nuclei and those that rely on the location of cytoplasmic proteins or cell membrane proteins. Exemplary segmentation methods are described in U.S. Patent Publication No. US-2002-0141631-A1 of Vaisberg et al., published Oct. 3, 2002, and titled “IMAGE ANALYSIS OF THE GOLGI COMPLEX,” U.S. Patent Publication No. US-2002-0154798-A1 of Cong et al. published Oct. 24, 2002 and titled “EXTRACTING SHAPE INFORMATION CONTAINED IN CELL IMAGES,” and U.S. patent application Ser. No. 11/651,885, filed Jan. 9, 2007, titled DOMAIN SEGMENTATION AND ANALYSIS, all of which are incorporated herein by reference for all purposes.

In one approach, individual nuclei are first located to identify discrete cells. Any suitable stain for DNA or histones may work for this purpose. Individual nuclei can be identified by performing, for example, a thresholding routine on images taken at a channel for the nuclear marker. After the nuclei are identified, cell boundaries can then be determined around each nucleus. In one embodiment, a non-specific marker for proteins such as Alexa 647 is used with an appropriate algorithm to identify cell boundaries. The assays described below include a DNA marker and a non-specific marker that may be used to facilitate segmentation.

Many features are defined on a per cell basis. More precisely, the features extracted on a per cell basis are typically aggregated over multiple cells in an image and provided as a statistical representation across all cells; e.g., a mean value across all cells in an image. In some cases, features are extracted from a limited domain within or near a cell in an image. For example, features may be extracted from a region bounded by a nucleus (e.g., identified by segmentation based on DNA or histone signal), a region identified as granules (e.g., particular gradient and size limitations), a region identified as cell peripheral regions (e.g., regions within certain distances of defined cell edges), etc. There are various reasons why a feature might be extracted from a sub-region within a cell. For example, changes in actin within the canaliculae may be a manifestation of cholestasis. Because canaliculae are often associated with inter-cellular junctions and reside in peripheral regions of cells, it may be desirable to employ a feature based on actin signal limited to peripheral or contact regions of cells. Further, sometimes a feature can be extracted most clearly when confined to a relatively thin layer of cytoplasm such as that which would be found overlying a cell nucleus. For example, some features pertaining to the texture or distribution of a cell component within the cytoplasm can be observed most clearly when taken from the portion of cytoplasm lying on top of a cell's nucleus.

Features defined within perimeter regions of cells may be particularly relevant to models of hepatotoxicity and associated pathologies. Examples of perimeter regions that may be employed include periphery regions, contact periphery regions, free periphery regions, and cell contact regions. These regions may be identified for the individual cells in the image. The periphery of a cell can be identified in the image as a subset of pixels inside the cell for which a mask with a predetermined size centered on each of the pixel covers at least one of the cell's boundary pixels. The contact periphery of a cell can be identified in the image as a subset of pixels inside the cell for which a mask with a predetermined size centered on each of the pixels covers at least one of the cell's boundary pixels and at least one boundary pixel of an adjacent cell. The free periphery of a cell can be identified in the image as a subset of pixels that are periphery pixels but not contact periphery pixels. Further discussion of these regions may be found in above-referenced U.S. patent application Ser. No. 11/651,885.

Many phenotypic features of interest are defined for or within nuclei or other organelles within cells, granules, and perimeter regions. Various pathologies may have signatures that are localized in the nuclei or cell perimeter regions for example. In such cases, it is desirable to consider phenotypic features from these regions. For example, certain conditions that interfere with cellular mitosis result in punctate or diffuse nuclei. Hence features such as the mean, standard deviation, and/or kurtosis of pixel intensity values located within a nuclei (identified by segmentation) can be useful in characterizing the condition of a cell with respect to a condition that interferes with mitosis.

Examples of markers used and a subset of features obtained in particular assays are given below. The list of features may represent a small subset of the features that are obtained across a group of assays, the total number of which (across all five assays in this example) is around 1500 in some embodiments. As indicated above, in certain embodiments, the features listed define the dimensions of the multi-dimensional space in which a distance from DMSO controls is calculated for each well (e.g., as shown in FIG. 4).

### Example 1

### Actin/Tubulin Assay

One example assay uses a tubulin marker (e.g., DM1-α), an actin marker (e.g, fluorescently labeled phalloidin), a DNA marker (e.g., Hoechst 33341) and a non-specific cellular protein marker (e.g., Alexa 647 nm succinimidyl ester). Tubulin and actin are cytoskeletal proteins, changes to the morphology or intensity of which may indicate hepatoxicity, including one or more hepatotoxic pathologies. Actin lines the canalicular structures which may be involved with bile transport. DNA and non-specific protein markers may be employed to facilitate segmentation of images into regions occupied by discrete cells as well as regions occupied by nuclei within cells. The following are a subset of features obtained in the actin/tubulin assay. In one example, the distances of wells from DMSO control are calculated using this subset of features:

mean area of the cells in the image

mean area of the nuclei in the image

mean axis ratio of the cells in the image

mean axis ratio of the nuclei in the image

mean circular variance of the cells in the image

mean kurtosis of the intensity of the Alexa signal of the cells in the image

mean kurtosis of the intensity of the Actin marker signal of the cells in the image

mean skewness of the intensity of the Alexa signal of the cells in the image

mean skewness of the intensity of the Actin marker signal of the cells in the image

mean total intensity of the Alexa signal of the cells in the image

mean total intensity of the Actin marker signal of the cells in the image

mean total intensity of the Actin marker signal in the contact periphery of the cells in the image

mean total intensity of the Actin marker signal in the free periphery of the cells in the image

Area may be determined from a pixel count within the boundary determined by segmentation (e.g. cell boundary or nucleus boundary). Axis ratio is calculated by fitting the cell or nucleus to an ellipse and calculating the ratio of the major and minor axes.

Circular variance represents the deviation of a particular shape or edge from a true circle. One goal is to distinguish elongated shapes from generally circular shapes. Shapes with a greater degree of elongation will have a larger value of circular variance. Briefly, circular variance is calculated from a centroid of an object or edge under consideration. The centroid (X, Y) represents the coordinate of the mean value of X and the mean value of Y in the edge under consideration. Once the centroid of an edge or closed region is identified, the radii between the centroid and each edge or boundary point are calculated. From these radii, a mean radius value r₀ is calculated for the edge under consideration. With this mean value and the individual radii, the circular variance can be calculated. Edges with a greater range in the value of their individual radii will give greater values of circular variance. Further discussion of this feature may be found in U.S. Patent Publication No. 20050273271 titled METHOD OF CHARACTERIZING CELL SHAPE, which is hereby incorporated by reference.

Kurtosis and skewness of the intensity are derived from fourth and third (respectively) moments of an intensity distribution. As the feature name suggests, mean kurtosis of the intensity of a particular marker within the cells of an image is determined by calculating the kurtosis of the intensity of the marker within each cell and taking the mean over all cells in the image. Mean skewness is similarly calculated. Mean total intensity is also calculated by determining the total intensity of the marker per cell or cell region (i.e., the contact and cell peripheries described above) and taking the mean over all cells or regions in the image.

### Example 2

### BSEP/MRP2

A second example assay uses a marker for Bile Salt Transporter protein (BSEP), a marker for Multidrug Resistance Protein 2 (MRP2), a DNA marker and a non-specific cellular protein marker (e.g., the Alexa 647 marker). BSEP and MRP2 are transporter proteins believed to be relevant to hepatotoxicity because both localize in the canaliculae, where bile transport may occur. Transport of bile across the canalicular membrane is mediated by BSEP, and it is believed that drug-induced cholestasis may be caused by direct inhibition of the BSEP transporter. MRP2 transports bile salts, and inhibition of its activity may also result, in intrahepatic cholestasis.

The following is a subset of features obtained in the BSEP/MRP2 assay. In one example, the distance of wells from DMSO control wells are calculated using these features:

mean granular area of the BSEP marker signal of the cells of the image

mean kurtosis of the intensity of the BSEP marker signal of the cells in the image

mean number of granules in cells as indicated by the BSEP marker signal in the image

mean moment 1 of the intensity of the BSEP marker signal of the cells in the image

mean moment 2 of the intensity of the BSEP marker signal of the cells in the image

mean skewness of the intensity of the BSEP marker signal of the cells in the image

mean total intensity of the BSEP marker signal of the cells in the image

mean total intensity of the BSEP marker signal in the contact periphery of the cells in the image

mean total intensity of the BSEP marker signal in the free periphery of the cells in the image

mean mean intensity of the BSEP marker signal of the cells in the image

mean mean intensity of the BSEP marker signal in the contact periphery of the cells in the image

mean mean intensity of the BSEP marker signal in the free periphery of the cells in the image

mean total granular intensity of the BSEP marker signal of the cells of the image

mean granular area of the MRP2 marker signal of the cells of the image

mean kurtosis of the intensity of the MRP2 marker signal of the cells in the image

mean number of granules in cells as indicated by the MRP2 marker signal in the image

mean moment 1 of the intensity of the MRP2 marker signal of the cells in the image

mean moment 2 of the intensity of the MRP2 marker signal of the cells in the image

mean skewness of the intensity of the MRP2 marker signal of the cells in the image

mean total intensity of the MRP2 marker signal of the cells in the image

mean total intensity of the MRP2 marker signal in the contact periphery of the cells in the image

mean total intensity of the MRP2 marker signal in the free periphery of the cells in the image

mean mean intensity of the MRP2 marker signal of the cells in the image

mean mean intensity of the MRP2 marker signal in the contact periphery of the cells in the image

mean mean intensity of the MRP2 marker signal in the free periphery of the cells in the image

mean total granular intensity of the MRP2 marker signal in the cells of the image

Mean granular area in the BSEP signal of cells of the image is determined by identifying the granules in the BSEP signal, calculating the total area of the granules per cell, and taking the mean area across all cells in the image. Similarly, number of granules and total granular intensity are determined by identifying the granules in the BSEP signal, and calculating the number of granules or total intensity of the granules on per cell basis and taking the mean across all cells.

Mean mean intensity of the BSEP marker is calculated by taking the mean intensity of the marker on a per cell or cell region basis, and taking the mean of the mean intensity across all cells or regions.

Moment 1 and moment 2 are additional measures of the moments of the distribution. Moment 1 is calculated using the following:

$${\sum }_{i = 1}^{N} {p}_{i} \sqrt{{( {x}_{i} - \overline{x} )}^{2} + {( {y}_{i} - \overline{y} )}^{2}}$$

and moment 2 is calculated by

$${\sum }_{i = 1}^{N} {p}_{i} [ {( {x}_{i} - \overline{x} )}^{2} + {( {y}_{i} - \overline{y} )}^{2} ]$$

where pᵢ is intensity of a pixel at coordinates (xᵢ, yᵢ) within an object (cell).

Other features (kurtosis, skewness, etc.) are as determined as discussed above with respect to the actin/tubulin assay.

### Example 3

### TGN/Cytochrome C

A third assay uses a Trans-Golgi Network (TGN) marker (e.g., TGN38), a cytochrome-C marker, a DNA marker and a non-specific cellular protein marker (e.g., the Alexa 647 marker). The Golgi network transports bile in hepatocytes. Its morphology is affected by bile transport. Hence, phenotypic features derived from markers for the trans-Golgi network are relevant to pathologies impacting (or impacted by) bile transport. Further, trafficking of the bile transporters BSEP and MRP2 occurs from the Golgi to the canalicular membrane, and disruption of this pathway may lead to alterations in Golgi morphology and intrahepatic cholestasis. Cytochrome-C is located in the mitochondrial matrix. Steatotic compounds affect lipid oxidation in the mitochondria. Inhibiting mitochondrial function may lead to an increase in intracellular neutral lipids and steatosis. Hence features derived from markers for mitochondrial proteins such as cytochrome-C may assist classifying stimuli inducing steatosis, cholestasis or other hepatotoxic pathologies.

The following is a subset of features obtained in the TGN/cytochrome-C assay. In one example, the distance of wells from DMSO control wells are calculated using these features:

mean kurtosis of the intensity of the TGN marker signal of the cells in the image

MOMENT1 of the intensity of the TGN marker signal of the cells in the image

MOMENT2 of the intensity of the TGN marker signal of the cells in the image

mean skewness of the intensity of the TGN marker signal of the cells in the image

mean total intensity of the TGN marker signal of the cells in the image

mean mean intensity of the TGN marker signal in the contact periphery of the cells in the image

mean mean intensity of the TGN marker signal in the free periphery of the cells in the image

mean mean intensity of the TGN marker signal of the cells in the image

mean mean intensity of the TGN marker signal in the contact periphery of the cells in the image

mean mean intensity of the TGN marker signal in the free periphery of the cells in the image

mean kurtosis of the intensity of the cytochrome-C marker signal of the cells in the image

mean skewness of the intensity of the cytochrome-C marker signal of the cells in the image

mean total intensity of the cytochrome-C marker signal of the cells in the image

mean mean intensity of the cytochrome-C marker signal in the contact periphery of the cells in the image

mean mean intensity of the cytochrome-C marker signal in the free periphery of the cells in the image

mean mean intensity of the cytochrome-C marker signal of the cells in the image

mean mean intensity of the cytochrome-C marker signal in the contact periphery of the cells in the image

mean mean intensity of the cytochrome-C marker signal in the free periphery of the cells in the image

These features are calculated as discussed above.

### Example 4

### BODIPY

A fourth assay uses a marker for lipids (e.g., BODIPY), a DNA marker and a non-specific cellular protein marker. Excessive accumulation of lipids and/or certain lipid morphologies are associated with hepatotoxicity, for example, steatosis. The following is a subset of features obtained in the BODIPY assay. In one example, the distance of wells from DMSO control wells are calculated using these features:

mean granular area of the BODIPY signal of the cells of the image

mean kurtosis of the intensity of the BODIPY signal of the cells in the image

mean number of granules in cells as indicated by the BODIPY signal in the image

mean total intensity of the BODIPY signal of the cells in the image

mean mean intensity of the BODIPY signal of the cells in the image

MOMENT1 of the intensity of the BODIPY marker signal of the cells in the image

mean total granular intensity of the BODIPY signal in the cells of the image

These features are calculated as discussed above.

### Example 5

### TRITC-DHPE

A fifth assay uses a fluorescently labeled phospholipid (e.g., TRITC-DHPE N-(6-tetramethylrhodaminethiocarbamoyl)-1,2-dihexadecanoyl-sn-glycero-3-phosphoethanolamine, triethylammonium salt), a DNA marker and a non-specific cellular protein marker. Phosholipidosis is an accumulation of phospholipids in lysosomes as lamellar bodies. Drug-induced phosholipidosis in hepatocytes can be measured by imaging the accumulation of DHPE-TRITC in lysosomes. As phospholipids are affected by phospholipidosis and possibly other hepatocyte pathologies, the DHPE marker can provide features useful in classifying hepatotoxicity. The following is a subset of features obtained in the DHPE assay. In one example, the distance of wells from DMSO control wells are calculated using these features:

mean kurtosis of the intensity of the DHPE signal of the nuclei in the image

mean total granular intensity of the DHPE signal of the cells of the image

These features are calculated as discussed above.

In the description of each of the above assays, a set of features was identified. These features define a multi-dimensional space within which results from tests employing compounds at a particular concentration (or more generally stimuli at particular levels) may be represented as single points. In the case where replicates are employed, a given compound/concentration has multiple points within this feature space. As explained previously, in certain embodiments, median values may be selected from among the data points produced from these replicates. Regardless or whether replicates are employed, a given compound/concentration data point may be assessed for “activity” by considering its position within the multi-dimensional phenotype space. As explained above, one measure of activity is a Euclidean distance from a central point in the feature space, which central point is associated little or no activity—e.g., the point representing a negative control produced by treating cells with DMSO or similar compound for example. Compound/concentrations producing data points separated by more than a “threshold” distance from the point of a negative control are deemed “active” and therefore made available for building a decision tree model or, in the reverse case, made available for classification by serving as inputs to such model. The threshold distances may be determined by empirically correlating level of activity (ability to induce pathology states) with numerical separation in the feature space. In certain embodiments, a compound/concentration is deemed active if its distance from the negative control is greater than a threshold distance in any assay (e.g., any of the five assays described above).

### MODEL EXAMPLES

Examples of models built using methods described herein are provided below. The models were built using data for about 200 compounds, each annotated as positive, negative or undefined for steatosis, cholestasis, phospholipidosis and hepatotoxicity. Multiple concentrations of each compound were applied to wells and the assays described above were performed. Distances from DMSO control wells were calculated for each well to identify the “active” wells using the features listed above. Features from one or more assays were then used to build models for each of the pathologies and overall hepatotoxicity.

Steatosis

Steatosis is a liver disorder marked by the accumulation of an abnormally large amount of fat within liver hepatocytes. The additional fat collects in vesicles that can be either large or small; when the vesicles are large the condition is known as macrovesicular steatosis and otherwise the condition is known as microvesicular steatosis. Steatosis is an important measure of liver function because the presence of steatosis can implicate a variety of serious medical conditions, such as hepatitis infection and liver disease due to chronic alcoholism.

In certain embodiments, computer-implemented methods of classifying a hepatocyte or population of hepatocytes according to whether they exhibit steatosis are provided. In certain embodiments the methods involve (a) receiving a set of phenotypic features of the hepatocyte or population of hepatocytes; (b) using at least a first subset of the set of phenotypic features of the hepatocyte or population of hepatocytes to determine whether the hepatocyte or hepatocytes exhibit a phenotype that is significantly different from a negative control phenotype; (c) if the hepatocyte or hepatocytes is determined in (b) to exhibit a phenotype that is significantly different from the negative control phenotype, providing a second subset of the set of phenotypic features from the hepatocyte or population of hepatocytes as an input to a model for classifying cells based on whether they exhibit steatosis; and (d) receiving a steatosis classification for the hepatocyte or population of hepatocytes as an output from the model.

Also provided are methods of producing a model for classifying hepatocytes according to a whether they exhibit steatosis, the method comprising: (a) receiving data points, each comprising (i) a set of phenotypic features of a hepatocyte or population of hepatocytes and (ii) and indication of whether steatosis is exhibited; (b) in a multi-dimensional phenotypic feature space, calculating a measure of difference, for each of the data points, between at least a first subset of the set of phenotypic features of the data point and corresponding phenotypic features of a negative control; (c) identifying those data points having measures of difference as calculated in (b) that are greater than a threshold value; and (d) using the data points identified in (c) to create a model for classifying hepatocytes according to whether they exhibit steatosis based on a second subset of the set of phenotypic features. In certain embodiments, the model is a decision tree. In certain embodiments, the model is an ensemble of decision trees. A decision tree model for steatosis may be produced by applying a random forest algorithm to the data points.

Models for steatosis may make use of various features calculated within the boundaries of whole cells, nuclei, peripheral regions of cells, as well as granules. Markers for lipids and proteins associated with canalicular structures may provide signal for various phenotypic features used as inputs for such models. Examples of suitable neutral lipid markers include BODIPY (available from Invitrogen, Carlsbad, Calif.) and Nile Red available from (available from Invitrogen, Carlsbad, Calif.). Examples of markers for canalicular structures include markers for BSEP, MDR2, and actin, for example. As one manifestation of steatosis is an accumulation of lipid vesicles within the cell, signal emanating from lipid markers, particularly signal having some granular morphology may be the basis for one or more features employed in decision tree models for steatosis. Markers for cytochrome C (including labeled cytochrome C itself) may also be useful for measuring steatosis, which may be caused by inhibiton of mitochondrial function.

In one example, random forest models for the prediction of steatosis were built as described above using a combination of all five assays described above. Variable selection was performed using a measure of decrease in accuracy that the random forest algorithm provides (see, e.g., Leo Breiman, “Random Forests—Random Features,” referenced above). The initial random forest model built using all five assays had 1481 features (independent variables). Successive models built based on the most important variables of the previous model had 103 and 13 variables, respectively. The variables used in the 13 variable model follow:

average ratio of the intensity of the BSEP signal to the MRP2 signal in the periphery region of the live cells in the image

mean granular area of the BODIPY signal of the cells of the image

mean total granular intensity of the BODIPY signal in the cells of the image

mean kurtosis of the intensity of the BODIPY signal of the nuclei in the image

mean moment 1 of the intensity of the BODIPY signal of the contact periphery of the cells in the image

mean number of granules in cells as indicated by the BODIPY signal in the image

mean skewness of the intensity of the BODIPY signal of the nuclei in the image

mean skewness of the intensity of the BODIPY signal in the free periphery of the cells in the image

mean standard deviation of the BODIPY signal of the nuclei in the image

mean total intensity of the BODIPY signal in the contact periphery of the cells in the image

mean total intensity of the BODIPY signal in the free periphery of the cells in the image

mean total intensity of the BODIPY signal in the periphery of the cells in the image

As can be seen from this list of features, all but one variable resulting from variable selection is a BODIPY feature. As BODIPY is a dye that tags neutral lipids, it may be particularly useful to characterize steatosis. Granularity of the lipids in the cells (as indicated by the granular area, intensity of the granule signal, and number of granular features) plays a significant role in the model; this may be because lipid vesicles (granules) are a manifestation of steatosis. Texture of the lipids in various regions of the cells (e.g., indicated by standard deviation, kurtosis and skewness of the BODIPY (neutral lipid) signal in one or more cell regions) also plays a role in this embodiment. Standard deviation, kurtosis, skewness, moment 1 and moment 2 of the BODIPY signal within the nuclei may indicate changes in accumulation and distribution of the stain, such as formation of granules. Lipid accumulation in the cell peripheries, represented by total intensity and skewness of the intensity of BODIPY in the peripheries may also be important in classifying stimuli for inducing steatosis. In some embodiments, the markers from which the phenotypic features are extracted include at least one marker for a neutral lipid and at least one marker for a phospholipid.

Features derived from non-lipid cell components can also be used in models for classifying cells/stimuli for steatosis. For example, features can be derived from one or more of a marker for a canalicular component, a marker for nuclear component, and a marker for general protein content within a cell. Further, as indicated, bile transport may also be important in characterizing steatosis. Therefore markers such as markers for BSEP and MRP2 may be employed in feature sets for steatosis models. In some embodiments, the markers from which phenotypic features are extracted include at least one marker for BSEP and at least one marker for MRP2. In the specific example presented here, the ratio of BSEP to MRP2 is a feature used in steatosis models.

Cholestasis

Cholestasis is characterized as inhibition of bile flow caused by a wide variety of mechanisms that involve elements of the biliary tree, including bile ducts, ductules, the basolateral or canalicular membrane, the tight junctions or pericanalicular network of the hepatocytes, the ATPase, and transporters of the hepatocytes' basolateral and canalicular plasma membranes. It may involve defects of the transport of bile acids from the sinusoidal blood into hepatocytes or from hepatocytes into bile. Any of these elements and mechanisms may give rise to phenotypic features used in models for classifying stimuli or cells based on cholestasis.

In certain embodiments, computer-implemented methods of classifying a hepatocyte or population of hepatocytes according to whether they exhibit cholestasis are provided. In certain embodiments the methods involve (a) receiving a set of phenotypic features of the hepatocyte or population of hepatocytes; (b) using at least a first subset of the set of phenotypic features of the hepatocyte or population of hepatocytes to determine whether the hepatocyte or hepatocytes exhibit a phenotype that is significantly different from a negative control phenotype; (c) if the hepatocyte or hepatocytes is determined in (b) to exhibit a phenotype that is significantly different from the negative control phenotype, providing a second subset of the set of phenotypic features from the hepatocyte or population of hepatocytes as an input to a model for classifying cells based on whether they exhibit cholestasis; and (d) receiving a cholestasis classification for the hepatocyte or population of hepatocytes as an output from the model.

Also provided are methods of producing a decision tree for classifying hepatocytes according to a whether they exhibit cholestasis, the method comprising: (a) receiving data points, each comprising (i) a set of phenotypic features of a hepatocyte or population of hepatocytes and (ii) and indication of whether cholestasis is exhibited; (b) in a multi-dimensional phenotypic feature space, calculating a measure of difference, for each of the data points, between at least a first subset of the set of phenotypic features of the data point and corresponding phenotypic features of a negative control; (c) identifying those data points having measures of difference as calculated in (b) that are greater than a threshold value; and (d) using the data points identified in (c) to create a model for classifying hepatocytes according to whether they exhibit cholestasis based on a second subset of the set of phenotypic features. In certain embodiments, the model is a decision tree. In certain embodiments, the model is an ensemble of decision trees. A decision tree model for cholestasis may be produced by applying a random forest algorithm to the data points.

Models for cholestasis may make use of various features calculated within the boundaries of whole cells, nuclei, peripheral regions of cells, as well as granules. In certain embodiments, at least one of the phenotypic features is extracted from segmented regions of the images corresponding to nuclei and/or peripheral regions of or within the cells. Cholestasis may be caused in some instances by damage to pericanalicular microfilaments. For example, cytochalasin B has been shown to produce a prompt arrest of bile flow in rats, thereby resulting in cholestatic injury. In addition, phalloidin causes an increase in filamentous F actin around canaliculi and tight junctions. Thus, changes in actin morphology or intensity features may be indicative of cholestatic injury. BSEP is the major bile salt transporter in the liver canalicular membrane. One of the physiological roles of MRP2 is to transport bilirubin glucuronides from liver into the bile. Thus, changes to BSEP and MRP2 may also be indicative of cholestatic injury. Further, the trans-Golgi network also plays a role in bile transport within hepatocytes. Hence, markers for any of BSEP, MRP2 (or other bile transport proteins), and the TGN are sometime employed in features for cholestasis models.

In some models, at least one of the one or more markers comprises a marker for a bile transport protein, a marker for general protein content within a cell, or a marker for a cytoskeletal component. In certain embodiments, the one or more markers includes markers for a Golgi component, general protein content within a cell, and/or a cytoskeletal component. In certain embodiments, the one or more markers includes a marker for general protein content within a cell, a marker for a cytoskeletal component, and a marker for a nuclear component. Regarding phenotypic features, at least one of the features may characterize canalicular structures at the periphery of hepatocytes. In certain embodiments, at least one of the features is derived from markers for one or more of MRP2, BSEP, TGN and cytochrome C.

Random forest models were built as described above using data from a combination of the Actin and BSEP/MRP2 assays, a combination of and the Actin and TGN/Cytochrome-C assays and the Actin assay alone. Variable selection was performed as discussed above for successive models. Some of the features used in the cholestasis models shown below are specific to live or dead cells. In certain embodiments, phenotypic features of cells obtained by the assay or assays may be used to determine if the cells are live or dead. See, e.g., the above-referenced patent application U.S. patent application Ser. No. 11/082,241, titled ASSAY FOR DISTINGUISHING LIVE AND DEAD CELLS (published as U.S. Patent Publication No. 20060014135) and U.S. patent application Ser. No. 11/355,258, filed Feb. 14, 2006 and titled ASSAY FOR DISTINGUISHING LIVE AND DEAD CELLS (published as U.S. Patent Publication 20070031818), hereby incorporated by reference in their entireties.

In one embodiment, the initial random forest model built using a combination of the Actin and BSEP/MRP2 assays had 973 features (independent variables). Successive models built based on the most important variables of the previous model had 145 and 21 variables, respectively. The variables used in the 21 variable model follow:

mean granular area of the Actin marker signal of the dead cells in the image

mean kurtosis of the BSEP marker signal in the contact periphery of the dead cells in the image

mean kurtosis of the intensity of the Alexa signal of the live cells in the image

mean kurtosis of the intensity of the Actin marker signal of the nuclei in the live cells in the image

mean kurtosis of the intensity of the Alexa signal of the cells in the image

number of contact peripheries in the image

number of granules in the dead cells as indicated by the MRP2 signal in the image

mean perimeter of the contact periphery of the cells in the image

mean R1 of the contact periphery of the live cells in the image

mean R2 of the contact periphery of the live cells in the image

mean SHARP of the BSEP marker signal in the contact regions of the dead cells in the image

mean SHARP of the Alexa signal in the nuclei of the live cells in the image

mean skewness of the intensity of the Alexa signal of the live cells in the image

mean skewness of the intensity of the Actin marker signal in the contact periphery of the live cells in the image

mean skewness of the intensity of the Alexa signal of the cells in the image

mean skewness of the intensity of the Actin marker signal in the contact periphery of the cells in the image

mean skewness of the intensity of the Actin marker signal of nuclei in the image

mean standard deviation of the intensity of the Hoechst signal in the dead cells of the image

mean standard deviation of the Actin marker signal in the nuclei of the image

mean total intensity of the Hoechst signal in the dead cells of the image

mean total intensity of the Hoechst signal in the nuclei of the dead cells in the image

R1 and R2 are morphological features related to moment 1 and moment 2. R1 is calculated using the following expression:

$${\sum }_{i = 1}^{N} {p}_{i} \sqrt{{( {x}_{i} - \overline{x} )}^{2} + {( {y}_{i} - \overline{y} )}^{2}}$$

and R2 is calculated using the following expression:

$${\sum }_{i = 1}^{N} [ {( {x}_{i} - \overline{x} )}^{2} + {( {y}_{i} - \overline{y} )}^{2} ]$$

where x and y are pixel coordinates within a segmented object, such as cell or cell component. Note that R1 and R2 are shape-based features; intensity need not be used in the calculation.

Sharp is a measure of the drop of the intensity at the edgy of an object. It may be calculated using the following expression:

$$\frac{1}{{N}_{c}} {\sum }_{i = 1}^{{N}_{c}} edge ( {x}_{i} , {y}_{i} ) ,$$

where N_{c} is the total number of edge pixels and edge(xᵢ, yᵢ) is obtained by the Marr-Hildreth edge detection operator.

Features based on the actin marker in the 21 variable model include granular area in the dead cells, R1 and R2, various features related texture including standard deviation, skewness and kurtosis of the intensity in the nuclei, skewness of the intensity in the contact periphery.

Features involving cellular protein (as marked by the Alexa marker in certain embodiments) and DNA (as marked by the Hoechst marker) are also important in this model. Features that may characterize the texture of cellular protein include kurtosis and skewness of the Alexa (non-specific protein) intensity. DNA-related features include intensity-related features of dead cells.

Only two BSEP/MRP2 features are provided among the 21 variables in the model: the number of MRP2 granules in the dead cells and SHARP of BSEP in the contact region. As mentioned, both BSEP and MRP2 are instrumental in the transport of bile within a cell; hence their role in some cholestasis models.

In another example, the initial random forest model built using a combination of the Actin and TGN/Cytochrome-C assays had 973 features (independent variables). Successive models built based on the most important variables of the previous model had 120 and 16 variables, respectively. The variables used in the 16 variable model follow:

mean granular area of the Actin marker signal of the dead cells in the image

mean kurtosis of the intensity of the Alexa signal of the live cells in the image

mean kurtosis of the intensity of the Actin marker signal of the nuclei in the live cells in the image

mean kurtosis of the intensity of the TGN marker signal of the periphery of the live cells in the image

mean kurtosis of the intensity of the Alexa signal of the cells in the image

number of contact peripheries in the image

mean R1 of the contact periphery of the live cells in the image

mean R2 of the contact periphery of the live cells in the image

mean SHARP of the Alexa signal in the nuclei of the live cells in the image

mean SHARP of the TGN signal in the nuclei of the live cells in the image

mean skewness of the intensity of the Alexa signal of the live cells in the image

mean skewness of the intensity of the TGN marker signal of the live cells in the image

mean skewness of the intensity of the Alexa signal of the cells in the image

mean total intensity of the Actin marker signal of the dead cells in the image

mean total intensity of the Hoechst signal of the dead cells in the image

mean total intensity of the Hoechst signal in the nuclei of the dead cells in the image

In yet another example, the initial random forest model built using the Actin assay alone had 575 features (independent variables). Successive models built based on the most important variables of the previous model had 79 and 10 variables, respectively. The variables used in the 10 variable model follow:

mean kurtosis of the intensity of the Alexa signal of the live cells in the image

mean kurtosis of the intensity of the DM1-α signal in the nuclei of the image

number of contact peripheries in the image

mean R1 of the Actin signal in the contact periphery of the live cells in the image

mean R2 of the Actin signal in the contact periphery of the live cells in the image

mean skewness of the intensity of the Alexa signal of the live cells in the image

mean skewness of the intensity of the Alexa signal of the cells in the image

mean skewness of the intensity of the Alexa signal in the nuclei of the cells in the image

mean total intensity of the Hoechst signal of the dead cells in the image

mean total intensity of the Hoechst signal in the nuclei of the dead cells in the image

Phospholipidosis

Another hepatotoxic pathology is phospholipidosis, a disorder that affects lipid storage, and particularly phospholipids. Phospholipids, which are structural components of mammalian cytoskeleton and cell membranes, accumulate in the cells. Phospholipid metabolism may be altered by drugs that interact with phospholipids or the enzymes that affect their metabolism. Cationic amphiphilic drugs (CADs), for example, may induce phospholipidosis. Phospholipidosis may also affect lysomsomal function. Lysosomes are subcellular organelles necessary for digestion of extracellular molecules, damaged or old cell parts and microorganisms. Lysosomes play an important role in detoxification of waste products.

In certain embodiments, the features derived from granules within cells feature prominently in models for phospholipidosis. Among such features are counts of lipid granules within hepatocytes, measures total lipid granule intensity within hepatocytes, and sizes of granules within hepatocytes (max, mean, etc.).

In certain embodiments, computer-implemented methods of classifying a hepatocyte or population of hepatocytes according to whether they exhibit phospholipidosis are provided. In certain embodiments the methods involve (a) receiving a set of phenotypic features of the hepatocyte or population of hepatocytes; (b) using at least a first subset of the set of phenotypic features of the hepatocyte or population of hepatocytes to determine whether the hepatocyte or hepatocytes exhibit a phenotype that is significantly different from a negative control phenotype; (c) if the hepatocyte or hepatocytes is determined in (b) to exhibit a phenotype that is significantly different from the negative control phenotype, providing a second subset of the set of phenotypic features from the hepatocyte or population of hepatocytes as an input to a model for classifying cells based on whether they exhibit phospholipidosis; and (d) receiving a phospholipidosis classification for the hepatocyte or population of hepatocytes as an output from the model.

Also provided are methods of producing a model for classifying hepatocytes according to a whether they exhibit phospholipidosis, the method comprising: (a) receiving data points, each comprising (i) a set of phenotypic features of a hepatocyte or population of hepatocytes and (ii) and indication of whether phospholipidosis is exhibited; (b) in a multi-dimensional phenotypic feature space, calculating a measure of difference, for each of the data points, between at least a first subset of the set of phenotypic features of the data point and corresponding phenotypic features of a negative control; (c) identifying those data points having measures of difference as calculated in (b) that are greater than a threshold value; and (d) using the data points identified in (c) to create a model for classifying hepatocytes according to whether they exhibit phospholipidosis based on a second subset of the set of phenotypic features. In certain embodiments, the model is a decision tree. In certain embodiments, the model is an ensemble of decision trees. A decision tree model for phospholipidosis may be produced by applying a random forest algorithm to the data points.

In certain embodiments, at least one of the one or more markers is a marker for general protein content within a cell or a marker for a phospholipid. In some cases, the markers from which the phenotypic features are extracted include at least one marker for DHPE (e.g., TRITC-DHPE). The phenotypic features employed in phospholipidosis models may be extracted from segmented regions of images corresponding to one or more of nuclei, granules, and peripheral regions within the cells. In some embodiments, a first phenotypic feature is extracted from segmented regions of the images corresponding to granules or peripheral regions within the cells, and a second phenotypic feature is extracted from segmented regions of the images corresponding to nuclei within the cells.

Random forest models were built as described above using the DHPE-TRITC assay alone. (An example of another assay for phospholipidosis is described in U.S. patent application Ser. No. 11/653,096 filed Jan. 12, 2007, which is hereby incorporated by reference). Variable selection was performed as discussed above for successive models. The initial random forest model built assays had 189 features (independent variables). Successive models built based on the most important variables of the previous model had 20 and 5 variables, respectively. The variables used in the 20 variable model follow:

mean granular area of the DHPE signal of the live cells in the image

mean skewness of the intensity of the DHPE signal of the nuclei in the image

mean kurtosis of the intensity of the DHPE signal of the nuclei in the image

mean kurtosis of the intensity of the DHPE signal in the contact periphery of the cells in the image

mean total granular intensity of the DHPE signal in the cells of the image

mean SHARP of the DHPE signal of the nuclei in the image

mean skewness of the intensity of the Alexa signal of the cells in the image

mean skewness of the intensity of the DHPE signal in the free periphery of the cells in the image

mean standard deviation of the intensity of the DHPE signal in the contact periphery of the cells in the image

mean skewness of the intensity of the DHPE signal in the cell contact regions in the image

mean skewness of the intensity of the DHPE signal of the cells in the image

mean number of granules in cells as indicated by the DHPE signal in the image

mean kurtosis of the intensity of the DHPE signal in the periphery of the cells in the image

mean kurtosis of the intensity of the DHPE signal of the cells in the image

mean standard deviation of the intensity of the DHPE signal in the periphery of the cells in the image

mean kurtosis of the intensity of the DHPE signal in the cell contact regions in the image

mean skewness of the intensity of the DHPE signal in the periphery of the cells in the image

mean standard deviation of the intensity of the DHPE signal of the cells in the image

mean skewness of the intensity of the DHPE signal in the contact periphery of the cells in the image

mean standard deviation of the intensity of the DHPE signal of the nuclei in the image

The first five features of those listed above were the variables used in the five variable model.

Hepatotoxicity Model

A stimulus applied to cells may be classified as hepatotoxic based on whether the stimulus induces a generic perturbation of the hepatocyte phenotype. Models for hepatotoxicity should be distinguished from models for specific pathologies such as cholestasis or steatosis. A perturbation classified as a hepatotoxic response may be a manifestation of any one or more pathologies including steatosis, phospholipidosis, cholestasis necrosis, carcinoma, PPAR, etc. Features from various assays may be used in an overall hepatotoxicity model. In a specific example described herein, hepatotoxicity models were built using a combination of the BSEP/MRP2, BODIPY and DHPE assays and a combination of the BSEP/MRP2 and BODIPY assays.

Various markers or combinations of markers may be employed in features used for models for hepatotoxicity. In certain embodiments, at least one of the markers is a marker for a cytoskeletal protein or structure, a marker for a canalicular component, a marker for an endocytic component, a marker for a mitochondrial component, a marker for nuclear component, a marker for a Golgi component, a marker for general protein content within a cell, or a marker for a lipid (neutral or phospholipid). In certain embodiments, the markers include markers for different types of lipids such as at least one marker for a neutral lipid and at least one marker for a phospholipid. In some embodiments, the markers include markers for two or more proteins associated with bile transport such as at least one marker for BSEP and at least one marker for MRP2. Note that the features employed in models for hepatotoxicity may be calculated within various boundaries identified by segmentation. Such boundaries may correspond to whole cells, nuclei, peripheral regions of cells, and/or granules.

An initial random forest model built using a combination of the BSEP/MRP2, BODIPY and DHPE assays had 868 features (independent variables). Thus, certain embodiments employ marker sets including at least markers for a neutral lipid, a phospholipid, and a bile transport protein. Other markers that may be included in this group include markers for a nuclear component and whole cellular protein. Successive models built based on the most important variables of the previous model had 172 and 29 variables, respectively. The variables used in the 29 variable model follow:

mean granular area of the BODIPY signal of the cells in the image

mean granular area of the DHPE signal of the cells in the image

mean total granular intensity of the BSEP marker signal of the live cells of the image

mean total granular intensity of the BODIPY signal of the cells of the image

mean total granular intensity of the BSEP marker signal of the cells of the image

mean total granular intensity of the DHPE signal of the cells of the image

mean kurtosis of the intensity of the Hoechst signal of the nuclei of the live cells in the image

mean kurtosis of the intensity of the Alexa signal of the live cells in the image

mean kurtosis of the intensity of the BODIPY signal in the contact periphery of the cells in the image

mean kurtosis of the intensity of the DHPE signal in the contact periphery of the cells in the image

mean kurtosis of the intensity of the BODIPY signal of the nuclei of the cells in the image

mean kurtosis of the intensity of the Hoechst signal of the nuclei of the cells in the image

mean major axis of the nuclei of the live cells in the image

mean major axis of the nuclei in the image

mean mean intensity of the MRP2 marker signal in the periphery of the live cells in the image

mean mean intensity of the Hoechst signal of the nuclei in the image

mean moment1 of the BODIPY signal of the nuclei in the image

number of “fuzzy” nuclei in the image based on Hoechst signal

number of cell contact regions of the live cells in the image

number of contact peripheries of the live cells in the image

number of cell contact regions in the image

mean number of granules in live cells as indicated by the BSEP marker signal in the image

mean number of granules in cells as indicated by the BODIPY signal in the image

mean number of granules in cells as indicated by the BSEP marker signal in the image

mean SHARP of the BSEP marker signal in the nuclei of the cells in the image

mean skewness of the intensity of the BODIPY signal of the nuclei in the image

mean standard deviation of the intensity of the Hoechst signal of the live cells in the image

mean standard deviation of the intensity of the MRP2 signal of the live cells in the image

mean standard deviation of the intensity of the BODIPY signal of the nuclei in the image

Note that an object is deemed to be “fuzzy” if the sharpness of the marker mask (e.g., DNA or Hoechst signal) is below a defined threshold. At least some of the “fuzzy” cells are dead and therefore have diffuse DNA staining.

Another initial random forest model built using a combination of the BSEP/MRP2 and BODIPY assays only had 815 features (independent variables). Successive models built based on the most important variables of the previous model had 140 and 23 variables, respectively. The variables used in the 23 variable model follow:

mean granular area of the BODIPY signal of the cells in the image

mean total granular intensity of the BSEP marker signal of the live cells of the image

mean total granular intensity of the BODIPY signal of the cells of the image

mean total granular intensity of the BSEP marker signal of the cells of the image

mean kurtosis of the intensity of the BSEP marker signal in the contact periphery of the live cells in the image

mean kurtosis of the intensity of the Hoechst signal in the nuclei of the live cells in the image

mean kurtosis of the intensity of the BODIPY signal in the nuclei of the cells in the image

mean kurtosis of the intensity of the Hoechst signal in the nuclei of the cells in the image

mean major axis of the nuclei in the image

mean mean intensity of the Hoechst signal of the live cells in the image

mean moment1 of the intensity of the BODIPY signal of the nuclei in the image

number of “fuzzy” nuclei in the image based on Hoechst signal

number of cell contact regions of the live cells in the image

number of contact peripheries of the live cells in the image

mean number of granules in live cells as indicated by the BSEP marker signal in the image

mean number of granules in cells as indicated by the BODIPY signal in the image

mean number of granules in cells as indicated by the BSEP marker signal in the image

mean SHARP of the BSEP marker signal in the nuclei of the cells in the image

mean skewness of the intensity of the MRP2 marker signal of the nuclei in the image

mean skewness of the intensity of the BODIPY signal of the nuclei in the image

mean standard deviation of the intensity of the Hoechst signal of the live cells in the image

mean standard deviation of the intensity of the BODIPY signal of the nuclei in the image

As illustrated in the above examples of hepatotoxicity models, lipid features may be taken within granule regions, nuclear regions, and cell peripheral regions. Features taken from bile transport proteins may likewise be taken within granule regions, nuclear regions, and cell peripheral regions. Further, some features are taken only from cells characterized as live cells. Some of these features are based on morphology. Others are based on intensity of signal or texture.

Image Capture and Imaging Apparatus

The assays described herein can be carried out in many different apparatuses. Generally, the cell samples are provided as discrete cell cultures on one or more support structures. Depending on the type of support structure, the cells may grow in two-dimensions or three-dimensions. Examples of support structures include bare plastic supports that include nutrients, glass surfaces, extra-cellular matrices such as collagen or Matrigel (available from BD Biosciences, San Jose, Calif.), etc. Such structures can be provided in multiwell plates, such as 24-, 96-, or 384-well assay plates (e.g., Costar plates (Corning Life Sciences, New York, N.Y.) among others). An assay plate is a collection of wells arranged in an array with each well holding multiple cells which are exposed to a stimulus or which provide a control sample. In other embodiments, single sample holders can be used instead of multi-well plates. Suitable culturing conditions and protocols for hepatocytes are described in U.S. Patent Publication No. 20050014217.

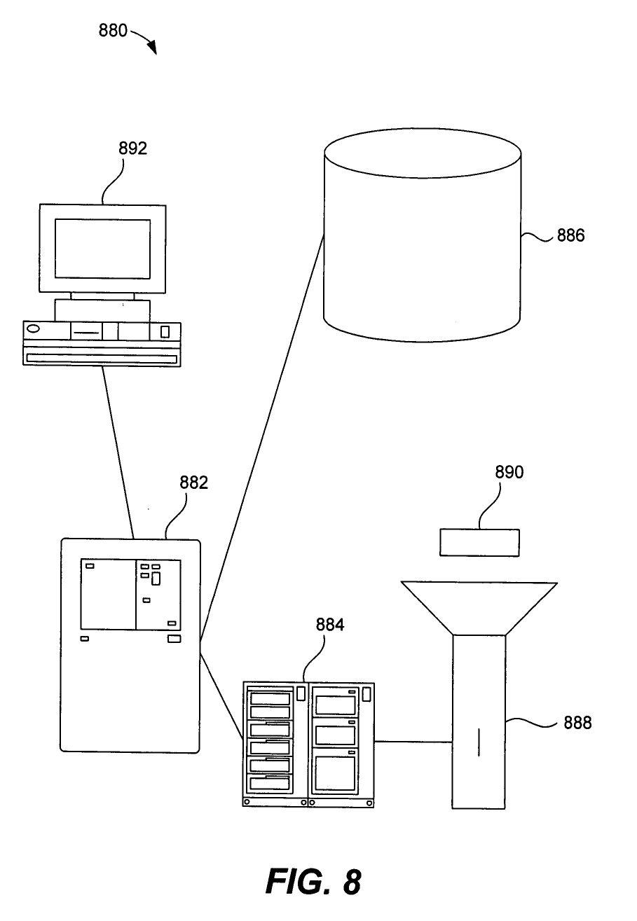

FIG. 8 shows a schematic block diagram of an image capture and image processing system 880 which can be used to capture and process the images of cells and store cell counts, phenotypic data, and other information used in assays of this invention. This diagram is merely a non-limiting example. The depicted system 80 includes a computing device 882, which is coupled to an image processor 884 and is coupled to a database 886. The image processor receives information from an image-capturing device 888, which includes an optical device for magnifying images of cells, such as a microscope. The image processor and image-capturing device can collectively be referred to as the imaging system herein. The image-capturing device obtains information from a plate 890, which includes a plurality of wells providing sites for groups of cells. The computing device 382 retrieves the information, which has been digitized, from the image-processing device and stores such information into the database 886.

A user interface device 892, which can be a personal computer, a work station, a network computer, a personal digital assistant, or the like, is coupled to the computing device. In the case of cells treated with a fluorescent marker, a collection of such cells is illuminated with light at an excitation frequency from a suitable light source such as a halogen-lamp, arc lamp or laser (not shown). A detector part of the image-capturing device is tuned to collect light at an emission frequency. Preferably this is a digital camera that is sensitive to light over a wide range of frequencies. One may use emission filters to control which light wavelengths hits the camera. Examples of suitable cameras are the Orca-100 from Hamamatsu (Hamamatsu City, Japan) or the CoolSNAP_{HQ}™ from Roper Scientific. The collected light is used to generate an image that highlights regions of high marker concentration.

The apparatus also includes a fluidics system for providing fluid to individual cell samples on the support. Such system can be employed to deliver a compound or other treatment to individual cell samples and to perform wash out on individual cell samples separately. An example is the fluidics system on the live cell imaging addition of the Axon ImageXpress (Axon Instruments/Molecular Devices Corporation, Union City, Calif.).

In one embodiment individual pipettes are provided for the individual wells of a support. Metered doses of a compound under investigation or a washing fluid are provided to each of the individual wells or to groups of individual wells as described above. The fluidics control system preferably allows precise control of the drug wash off timing and flow conditions. In certain embodiments, a key is to ensure thorough exchange of the compound, without also dislodging viable cells. And in some cases, it may be desirable that no cells, even dead cells, be washed away. So precise control of fluid force and turbulence can be important. To this end, the fluidics control system preferably allows fine control of fluid flow rates, delivery times, aspiration rates, and separation distance of the pipette or other delivery nozzle from the wells. A flexible fluidics system is desirable in any apparatus that is used to carry out different types of assay, as some treatments are more difficult to wash away than other, and some cells are more sensitive to wash out conditions than others. In situations where the cells are extremely sensitive and the treatment is difficult to remove, the apparatus may include a semipermeable covering over the individual cell samples, to allow washing fluid to penetrate to the cells but prevent the cells themselves from being washed away.

The apparatus may also allow careful control of illumination conditions. Obviously when fluorescent markers are used the apparatus must be able to illuminate at appropriate excitation frequencies and capture radiation at the signature emission frequencies. However, it may also be important to ensure that the illumination conditions do not kill cells. Phototoxicity is a consideration. In a time-lapse assay, imaging parameters to be optimized include the intensity of illumination (which may dictate magnification) and the frequency at which individual images are captured. Again, different types of cells and different treatment regimens lead to different levels of sensitivity. So systems allowing flexible illumination conditions are generally preferred.

Other apparatus features include, optionally, mechanisms for controlling the environment in which the cells grow. Thus, the apparatus may include sub-systems for monitoring and controlling temperature and the atmospheric composition (e.g., carbon dioxide levels).

Image Processing and Analysis

As indicated, the images used as the starting point for the methods of this invention are obtained from cells that have been specially treated and/or imaged under conditions that contrast the cellular components of interest with other cellular components and the background of the image. These images may be processed in an automated manner employing image analysis software.

The individual images are processed using, for example, image correction and image processing techniques in order to extract the appropriate cellular features. Initially, the images can be corrected to remove artifacts introduced by the image capture system and to remove background. As an alternative to correction, “quality control algorithms” may be employed to discard image data based on, for example, poor exposure, focus failures, foreign objects, and other imaging failures. In one embodiment, problem images can be identified by abnormal intensities and/or spatial statistics.

In a specific embodiment, a correction algorithm may correct for changing light conditions, positions of wells, etc. In one example, a noise reduction technique such as median filtering is employed. Then a correction for spatial differences in intensity may be employed. The spatial correction may comprise a separate model for each image (or group of images). These models may be generated by separately summing or averaging all pixel values in the x-direction for each value of y and then separately summing or averaging all pixel values in the y direction for each value of x. In this manner, a parabolic set of correction values is generated for the image or images under consideration. Applying the correction values to the image adjusts for optical system non-linearities, mis-positioning of wells during imaging, etc. Note that different correction techniques and quality control algorithms can be carried out depending on the type of imaging that is used, e.g. brightfield, confocal or deconvolution.

After image correction, a segmentation process is carried out to identify individual objects within the images. If these objects represent single cells, they can be counted to give cell counts at the various phases of the process as described above. Generally, segmentation allows feature extraction on a cell-by-cell basis. Segmentation identifies discrete regions of an image that include only those pixels where the components of a single cell are deemed to be present. Thus, each representation resulting from segmentation is a bounded collection of pixels associated with one or more features characterizing a single cell.

Segmentation can be accomplished in numerous ways as indicated elsewhere herein. These include use of watershed algorithms and techniques that identify separate nuclei. In many cases, the segmentation process identifies “edges” (locations in the images where there is a sudden change in pixel intensity) and then looks for closed connected edges in order to identify an object.

At every combination of dose and compound, one or more images are obtained. As indicated, these images are used to extract various parameter values for cellular features of relevance to a biological phenomenon of interest. Generally a given image of a cell, as represented by one or more markers, can be analyzed in isolation or in combination with other images of the same cell (as provided by different markers), to obtain any number of image features.

It will be appreciated that any simple or complex cellular feature than can be derived from the images is suitable for use in the present invention and that the invention is not to be limited to the specific examples given, nor to the specific sequence of actions, which is merely by way of an illustrative example. The result of this processing can be thousands or tens of thousands of cellular features derived from each of the treated wells and control wells.

After the features have been extracted from the image they may be stored in database 386, and analysis of the features is carried out in order to assess the effect of the treatment on the cells.

In general, cells from a well are evaluated and some statistics for that well, e.g. the averages of various properties, are calculated. In some cases, the same quantity is obtained for replicate wells (e.g., the other five wells when the experiment is replicated six times) and statistics are computed on those statistics for the replicate wells in order to aggregate (e.g. obtain the median of the average value mentioned above). However, averaging is not necessary and instead cell level information can be used, and have all further computations to be based on cell level information. Hence, for each compound/dose/cell line/time point/marker set/etc. there would be thousands of data points.

In assays of this invention, it may be desirable to characterize the effect of the stimulus as a function of the dose or level of that stimulus. Cell counts and various phenotypic traits may be analyzed as a function of concentration (or other level of stimulus). When replicates or multiple cell lines are used, an average simple cellular feature can be obtained for each cell line at each dose level. However, it is not necessary to calculate averages over cells. Also, other statistical measures can be used such as the median, specific quantiles, and standard deviations. Further, the statistical properties need not be calculated over all cells, but can be calculated over a sub-population of cells, for example over the sub-group of interphase cells, or the sub-group of cells that are arrested in mitosis for a period of time prior to compound wash out (e.g., 3-4 hours). In that case, a cell cycle related classification of the cells is carried out prior to summarizing or averaging the cell feature values.

The characterization of the stimulus (in terms of cell count, morphological effects, etc.) is sometimes referred to as a “path” or “response curve.” Mathematically, the path is made up of multiple points, each at a different level of the stimulus. Each of these points is comprised of one or more parameters describing some aspect of a cell or collection of cells. In the sense that each point or signature in the path may contain more than one piece of information about a cell, the points may be viewed as arrays, vectors, matrices, etc. Individual stimulus-response paths can be compared based on similarity of trajectory, distance between paths or segments thereof. In one example, the dose response can be compared across multiple cell lines, with each cell line providing its own dose-response path. Such comparisons provide meaningful information about drug selectivity, potency, mechanism of action, etc.

One biological classification having application in this invention is whether a cell is alive or dead, and particularly whether a cell is apoptotic or not. Apoptotic cells may be identified by various techniques. Apoptosis is characterized by a pathway that includes changes in certain membrane proteins, depolarization of the mitochondrial membrane, release of cytochrome C from mitochondria, condensation, fragmentation and granularization of the nuclei, and breakdown of various nuclear and cellular proteins including actin, and microtubules. Many of these manifestations can be identified by image analysis. Examples include exposure of phosphatidyl serines on membrane proteins, the migration of cytochrome c from the mitrochondria into other regions of the cell, changes of mitochondrial membrane potential, and condensation, fragmentation and granularization of the nuclei.

In certain embodiments, cells under investigation are cultured with a marker that selectively penetrates into dead cells (and is excluded from live cells), where it marks one or more features in the cytoplasm and/or nucleus. An example of such marker is propidium iodide, which penetrates the membrane of only those cells that have died.

Another property of cells undergoing apoptosis is that they tend to become loosely attached to a substrate. Both cytoplasm shrinkage and loss of attachment may be a result of cytoskeleton damage by caspases. This property can be detected by exposing the culture to a treatment that will tend to dislodge and remove loosely attached cells. As indicated, some embodiments of the invention employ careful washing to accomplish this. The level of apoptosis has been found to correlate well to a “washout coefficient” based on cell counts in washed and unwashed cultures exposed to a stimulus suspected of inducing apoptosis; e.g., (cc(unwashed)−cc(washed))/cc(unwashed).

Computational Systems

Methods, devices, systems and apparatus provided herein can be implemented in digital electronic circuitry, or in computer hardware, firmware, software, or in combinations of them. Apparatus can be implemented in a computer program product tangibly embodied in a machine-readable storage device for execution by a programmable processor; and aspects of the methods provided can be performed by a programmable processor executing a program of instructions to perform, e.g., clustering training set data, generating random forest models from clusters of training set data, operating on input data (e.g., images in a stack), extracting cellular phenotypic features from images, predicting outcomes and/or classifying responses (e.g., mechanisms of action for certain compounds) using models having as inputs phenotypic characteristics of cells, identifying cellular boundary regions, and other processing algorithms.

Methods provided herein can be implemented in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

To provide for interaction with a user, methods can be implemented on a computer system having a display device such as a monitor or LCD screen for displaying information to the user. The user can provide input to the computer system through various input devices such as a keyboard and a pointing device, such as a mouse, a trackball, a microphone, a touch-sensitive display, a transducer card reader, a magnetic or paper tape reader, a tablet, a stylus, a voice or handwriting recognizer, or any other well-known input device such as, of course, other computers. The computer system can be programmed to provide a graphical user interface through which computer programs interact with users.

Finally, the processor optionally can be coupled to a computer or telecommunications network, for example, an Internet network, or an intranet network, using a network connection, through which the processor can receive information from the network, or might output information to the network in the course of performing the above-described method steps. Such information, which is often represented as a sequence of instructions to be executed using the processor, may be received from and outputted to the network, for example, in the form of a computer data signal embodied in a carrier wave. The above-described devices and materials will be familiar to those of skill in the computer hardware and software arts.

It should be noted that methods and other aspects provided may employ various computer-implemented operations involving data stored in computer systems. These operations include, but are not limited to, those requiring physical manipulation of physical quantities. Usually, though not necessarily, these quantities take the form of electrical or magnetic signals capable of being stored, transferred, combined, compared, and otherwise manipulated. The operations described herein that may form part of the methods described are useful machine operations. The manipulations performed are often referred to in terms, such as, producing, identifying, running, determining, comparing, executing, downloading, or detecting. It is sometimes convenient, principally for reasons of common usage, to refer to these electrical or magnetic signals as bits, values, elements, variables, characters, data, or the like. It should remembered however, that all of these and similar terms are to be associated with the appropriate physical quantities and are merely convenient labels applied to these quantities.

Also provided are devices, systems and apparatus for performing the aforementioned operations. The system may be specially constructed for the required purposes, or it may be a general-purpose computer selectively activated or configured by a computer program stored in the computer. The processes presented above are not inherently related to any particular computer or other computing apparatus. Various general-purpose computers may be used with programs written in accordance with the teachings herein, or, alternatively, it may be more convenient to construct a more specialized computer system to perform the required operations.

The above discussion has focused on hepatocytes and hepatotoxic responses. However, the description provided herein extends beyond hepatotoxicity to toxicity and pathologies in a variety of other cell lines, cell types, and tissues.

Although the above has provided a general description according to specific processes, various modifications can be made without departing from the spirit and/or scope of the description provided. Those of ordinary skill in the art will recognize other variations, modifications, and alternatives.

What is claimed is:

1. A method of generating a model for classifying hepatotoxicity of a stimulus applied to a test population of cells, the method comprising:
(a) receiving a data set comprising values for dependent variables associated with stimuli;
(b) preparing a set of cell populations treated with said stimuli;
(c) identifying a subset of the treated cell populations to be used in generating a model for classifying hepatotoxicity of stimuli; and
(d) generating said model from phenotypic data associated with the subset,
wherein the model is provided to classify stimuli based on hepatotoxic effects they produce in a test population of cells.

2. The method of claim 1 wherein the dependent variables indicate whether the stimuli induce at least one of cholestasis, phospholipidosis and steatosis.

3. The method of claim 1 wherein the set of cell populations in (b) comprises cell populations treated with multiple levels of the stimuli.

4. The method of claim 1 wherein (c) comprises comparing one or more phenotypic features of the cells in the set with phenotypic features of one or more control populations.

5. The method of claim 4 wherein (c) further comprises calculating one or more multi-dimensional distances from the control populations in phenotype space for each cell population in the set, wherein the dimensions are phenotypic features.

6. The method of claim 1 wherein (c) comprises performing one or more assays to determine phenotypic features of the cell populations in the set.

7. The method of claim 1 wherein the data associated with the subset comprises independent and dependent variables associated with the cell populations in the subset.

8. The method of claim 7 wherein the independent variables comprise at least one of: the intensities of a marker within the cell populations, the morphologies of a marker within the cell populations.

9. The method of claim 1 wherein (d) comprises:
receiving a training set comprising values for independent and dependent variables associated with cell populations in the subset;
randomly selecting, with replacement, cell populations to construct multiple bootstrap samples of the size of the training set; and
generating a random forest model for each bootstrap sample, wherein an ensemble of the random forest models is provided to classify the test population.

10. The method of claim 9, wherein (d) further comprises clustering the training set such that clusters of cell populations based on types of applied stimulus are selected with replacement to generate the bootstrap samples.

11. The method of claim 1 wherein generating the model comprises generating a random forest model by growing an unpruned decision tree by randomly selecting a subset of independent variables at each node and choosing the variable that produces the best split for that node.

12. A method of classifying hepatotoxicity of a stimulus, the method comprising:
a) providing a model generated according to claim 1; and
b) applying the independent variables to the model to produce a hepatotoxicity classification of the stimulus.

13. A computer program product comprising a machine readable medium on which is provided program instructions for generating a model for classifying hepatotoxicity of a stimulus applied to a test population of cells, the instructions comprising:
(a) code for receiving a data set comprising values for dependent variables associated with stimuli;
(b) code for receiving information about a set of cell populations treated with said stimuli;
(c) code for identifying a subset of the treated cell populations to be used in generating a model for classifying hepatotoxicity of stimuli; and
(d) code for generating said model from phenotypic data associated with the subset, wherein the model is provided to classify stimuli based on hepatotoxic effects they produce in a test population of cells.

14. The computer program product of claim 13 wherein the dependent variables indicate whether the stimuli induce at least one of cholestasis, phospholipidosis and steatosis.

15. The computer program product of claim 13 wherein the set of cell populations in (b) comprises cell populations treated with multiple levels of the stimuli.

16. The computer program product of claim 13 wherein (c) comprises code for comparing one or more phenotypic features of the cells in the set with phenotypic features of one or more control populations.

17. The computer program product of claim 16 wherein (c) further comprises code for calculating one or more multi-dimensional distances from the control populations in phenotype space for each cell population in the set, wherein the dimensions are phenotypic features.

18. The computer program product of claim 13 wherein (c) comprises code for receiving information determined from one or more assays to determine phenotypic features of the cell populations in the set.

19. The computer program product of claim 13 wherein the data associated with the subset comprises independent and dependent variables associated with the cell populations in the subset.

20. The computer program product of claim 19 wherein the independent variables comprise at least one of: the intensities of a marker within the cell populations, the morphologies of a marker within the cell populations.

21. The computer program product of claim 13 wherein (d) comprises:
code for receiving a training set comprising values for independent and dependent variables associated with cell populations in the subset;
code for randomly selecting, with replacement, cell populations to construct multiple bootstrap samples of the size of the training set; and
code for generating a random forest model for each bootstrap sample, wherein an ensemble of the random forest models is provided to classify the test population.

22. The computer program product of claim 20, wherein (d) further comprises code for clustering the training set such that clusters of cell populations based on types of applied stimulus are selected with replacement to generate the bootstrap samples.

23. The computer program product of claim 13 wherein code for generating the model comprises code for generating a random forest model by growing an unpruned decision tree by randomly selecting a subset of independent variables at each node and choosing the variable that produces the best split for that node.

24. A computer program product comprising a machine readable medium on which is provided program instructions for classifying hepatotoxicity of a stimulus, the instructions comprising:
a) code for generating a model according to claim 13; and
b) code for applying the independent variables to the model to produce a hepatotoxicity classification of the stimulus.

25. A computer implemented method of classifying a cell or population of cells by pathology or toxic response, the method comprising:
(a) receiving a set of phenotypic features of the cell or population of cells;
(b) in a multi-dimensional phenotypic feature space, calculating a measure of difference between at least a first subset of the set of phenotypic features of the cell or population of cells and corresponding phenotypic features of a negative control;
(c) determining that the measure of difference calculated in (b) is greater than a threshold value;
(d) providing a second subset of the set of phenotypic features from the cell or population of cells as an input to a model for classifying cells based on pathology or toxic response; and
(e) receiving a pathology or toxic response classification for the cell or population of cells as an output from the model.

26. The method of claim 25, wherein the first subset of phenotypic features and the second subset of phenotypic features are different.

27. The method of claim 25, wherein at least one of the first and second subsets is identical to the set of phenotypic features.

28. The method of claim 25, wherein the pathology or toxic response is associated with hepatocytes and the cell or population of cells comprises a hepatocyte or population of hepatocytes.

29. The method of claim 25, wherein the phenotypic features are obtained automatically by image analysis.

30. The method of claim 25, wherein the measure of difference calculated in (b) is a Euclidean or Manhattan distance.

31. The method of claim 25, wherein the pathology classification is one or more of cholestasis, steatosis, and phospholipidosis.

32. The method of claim 25, wherein the model for classifying cells based on pathology or toxic response comprises a decision tree.

33. A method of producing a model for classifying cells according to a pathology or toxic response, the method comprising:
(a) receiving data points, each comprising (i) a set of phenotypic features of a cell or population of cells and (ii) an indication of whether the pathology or toxic response is present;
(b) in a multi-dimensional phenotypic feature space, calculating a measure of difference for each of the data points, between at least a first subset of the set of phenotypic features of the data point and corresponding phenotypic features of a negative control;
(c) identifying those data points having a measure of difference as calculated in (b) that is greater than a threshold value; and
(d) applying an algorithm to the data points identified in (c) to thereby create a model for classifying cells according to the pathology or toxic response based on a second subset of the set of phenotypic features.

34. The method of claim 33, wherein the first subset of phenotypic features and the second subset of phenotypic features are different.

35. The method of claim 33, wherein at least one of the first and second subsets is identical to the set of phenotypic features.

36. The method of claim 33, wherein the pathology or toxic response is associated with hepatocytes and the cell or population of cells comprises a hepatocyte or population of hepatocytes.

37. The method of claim 33, wherein the phenotypic features are obtained automatically by image analysis.

38. The method of claim 33, wherein the measure of difference calculated in (b) is a Euclidean distance or a Manhattan distance.

39. The method of claim 33, wherein the pathology classification is one or more of cholestasis, steatosis, and phospholipidosis.

40. The method of claim 33, wherein the model for classifying cells based on pathology or toxic response comprises a decision tree.

41. The method of claim 33, wherein applying an algorithm to the data points to thereby create a model comprises applying a random forest algorithm to the data points.

42. A computer program product comprising a machine readable medium on which is provided program instructions for classifying a cell or population of cells by pathology or toxic response, the instructions comprising:
(a) code for receiving a set of phenotypic features of the cell or population of cells;
(b) code for, in a multi-dimensional phenotypic feature space, calculating a measure of difference between at least a first subset of the set of phenotypic features of the cell or population of cells and corresponding phenotypic features of a negative control;
(c) code for determining that the measure of difference calculated in (b) is greater than a threshold value;
(d) code for providing a second subset of the set of phenotypic features from the cell or population of cells as an input to a model for classifying cells based on pathology or toxic response; and
(e) code for receiving a pathology or toxic response classification for the cell or population of cells as an output from the model.

43. The computer program product of claim 42, wherein the first subset of phenotypic features and the second subset of phenotypic features are different.

44. The computer program product of claim 42, wherein at least one of the first and second subsets is identical to the set of phenotypic features.

45. The computer program product of claim 42, wherein the pathology or toxic response is associated with hepatocytes and the cell or population of cells comprises a hepatocyte or population of hepatocytes.

46. The computer program product of claim 42, wherein the phenotypic features are obtained automatically by image analysis.

47. The computer program product of claim 42, wherein the measure of difference calculated in (b) is a Euclidean distance or a Manhattan distance.

48. The computer program product of claim 42, wherein the pathology classification is one or more of cholestasis, steatosis, and phospholipidosis.

49. The computer program product of claim 42, wherein the model for classifying cells based on pathology or toxic response comprises a decision tree.

50. A computer program product comprising a machine readable medium on which is provided program instructions for producing a model for classifying cells according to a pathology or toxic response, the instructions comprising:
(a) code for receiving data points, each comprising (i) a set of phenotypic features of a cell or population of cells and (ii) an indication of whether the pathology or toxic response is present;
(b) code for, in a multi-dimensional phenotypic feature space, calculating a measure of difference for each of the data points, between at least a first subset of the set of phenotypic features of the data point and corresponding phenotypic features of a negative control;
(c) code for identifying those data points having a measure of difference as calculated in (b) that is greater than a threshold value; and
(d) code for applying an algorithm to the data points identified in (c) to thereby create a model for classifying cells according to the pathology or toxic response based on a second subset of the set of phenotypic features.

51. The computer program product of claim 50 wherein the first subset of phenotypic features and the second subset of phenotypic features are different.

52. The computer program product of claim 50, wherein at least one of the first and second subsets is identical to the set of phenotypic features.

53. The computer program product of claim 50, wherein the pathology or toxic response is associated with hepatocytes and the cell or population of cells comprises a hepatocyte or population of hepatocytes.

54. The computer program product of claim 50, wherein the phenotypic features are obtained automatically by image analysis.

55. The computer program product of claim 50, wherein the measure of difference calculated in (b) is a Euclidean distance or a Manhattan distance.

56. The computer program product of claim 50, wherein the pathology classification is one or more of cholestasis, steatosis, and phospholipidosis.

57. The computer program product of claim 50, wherein the model for classifying cells based on pathology or toxic response comprises a decision tree.

58. The computer program product of claim 50, wherein code for applying an algorithm to the data points to thereby create a model comprises code for applying a random forest algorithm to the data points.

59. A method of producing a model for classifying stimuli based on hepatotoxicity, the method comprising:
receiving images of hepatocytes which have been exposed to stimuli and treated with one or more markers for cellular components in the hepatocytes;
automatically extracting two or more phenotypic features from the one or more markers in the images, wherein at least one of the features is extracted from segmented regions within the hepatocyte images, which segmented regions correspond to granules and/or peripheral regions within the hepatocytes;
providing a training set comprising data points, each data point comprising (i) the two or more phenotypic features and (ii) an indication of the presence or absence of hepatotoxicity in the stimuli applied to the hepatocytes from which the phenotypic features were obtained; and
generating a model from the training set, the model classifying stimuli according to whether they are hepatotoxic.

60. The method of claim 59, wherein at least one of the one or more markers comprises a marker for a cytoskeletal protein or structure, a marker for a canalicular component, a marker for an endocytic component, a marker for a mitochondrial component, a marker for nuclear component, a marker for a Golgi component, a marker for general protein content within a cell, or a marker for a lipid.

61. The method of claim 59, wherein the markers from which the phenotypic features are extracted comprise at least one marker for a neutral lipid and at least one marker for a phospholipid.

62. The method of claim 59, wherein the markers from which the phenotypic features are extracted comprise at least one marker for BSEP and at least one marker for MRP2.

63. The method of claim 59, wherein at least one of the features is extracted from segmented regions corresponding to nuclei within the hepatocytes.

64. The method of claim 59, wherein at least one of the features is extracted from segmented regions corresponding to whole cells.

65. The method of claim 59, wherein the model for classifying stimuli comprises a decision tree.

66. The method of claim 59, wherein generating the model comprises applying a random forest algorithm to the training set.

67. The method of claim 59, wherein providing the training set comprises selecting data points for stimuli having a level of activity sufficient to induce a change greater than a defined magnitude in at least some phenotypic features.

68. The method of claim 67, wherein selecting data points is performed for stimuli indicated to exhibit hepatotoxicity.

69. The method of claim 67, wherein selecting data points is performed for stimuli indicated to exhibit hepatotoxicity and for stimuli not indicated to exhibit hepatotoxicity.

70. A computer implemented method of classifying a stimulus according to whether it is hepatotoxic, the method comprising:
receiving at least one image of hepatocytes which have been exposed to the stimulus and treated with one or more markers for cellular components in the hepatocytes;
automatically extracting two or more phenotypic features from the one or more markers in the image, wherein at least one of the features is extracted from segmented regions within the hepatocyte images, which segmented regions correspond to granules and/or peripheral regions within the hepatocytes;
applying the two or more phenotypic features to a model for classifying stimuli according to whether they are hepatotoxic; and
receiving a hepatatotoxicity classification for the stimulus as an output from the model.

71. The method of claim 70, wherein at least one of the one or more markers comprises a marker for a cytoskeletal protein or structure, a marker for a canalicular component, a marker for an endocytic component, a marker for a mitochondrial component, a marker for nuclear component, a marker for a Golgi component, a marker for general protein content within a cell, or a marker for a lipid.

72. The method of claim 70, wherein the markers from which the phenotypic features are extracted comprise at least one marker for a neutral lipid and at least one marker for a phospholipid.

73. The method of claim 70, wherein the markers from which the phenotypic features are extracted comprise at least one marker for BSEP and at least one marker for MRP2.

74. The method of claim 70, wherein at least one of the features is extracted from segmented regions corresponding to nuclei within the hepatocytes.

75. The method of claim 70, wherein at least one of the features is extracted from segmented regions corresponding to whole cells.

76. The method of claim 70, wherein the model for classifying stimuli comprises a decision tree.

77. The method of claim 70, further comprising, prior to applying the two or more phenotypic features to a model, determining that the stimulus has a level of activity sufficient to induce a change greater than a defined magnitude in at least some phenotypic features.

78. The method of claim 70, wherein the method is performed without determining whether the stimulus has a level of activity sufficient to induce a change greater than a defined magnitude in at least some phenotypic features.

79. A computer program product comprising a machine readable medium on which is provided program instructions for producing a model for classifying stimuli based on hepatotoxicity, the instructions comprising:
code for receiving images of hepatocytes which have been exposed to stimuli and treated with one or more markers for cellular components in the hepatocytes;
code for automatically extracting two or more phenotypic features from the one or more markers in the images, wherein at least one of the features is extracted from segmented regions within the hepatocyte images, which segmented regions correspond to granules and/or peripheral regions within the hepatocytes;
code for providing a training set comprising data points, each data point comprising (i) the two or more phenotypic features and (ii) an indication of the presence or absence of hepatotoxicity in the stimuli applied to the hepatocytes from which the phenotypic features were obtained; and
code for generating a model from the training set, the model classifying stimuli according to whether they are hepatotoxic.

80. The computer program product of claim 79, wherein at least one of the one or more markers comprises a marker for a cytoskeletal protein or structure, a marker for a canalicular component, a marker for an endocytic component, a marker for a mitochondrial component, a marker for nuclear component, a marker for a Golgi component, a marker for general protein content within a cell, or a marker for a lipid.

81. The computer program product of claim 79, wherein the markers from which the phenotypic features are extracted comprise at least one marker for a neutral lipid and at least one marker for a phospholipid.

82. The computer program product of claim 79, wherein the markers from which the phenotypic features are extracted comprise at least one marker for BSEP and at least one marker for MRP2.

83. The computer program product of claim 79, wherein at least one of the features is extracted from segmented regions corresponding to nuclei within the hepatocytes.

84. The computer program product of claim 79, wherein at least one of the features is extracted from segmented regions corresponding to whole cells.

85. The computer program product of claim 79, wherein the model for classifying stimuli comprises a decision tree.

86. The computer program product of claim 79, wherein code for generating the model comprises code for applying a random forest algorithm to the training set.

87. The computer program product of claim 79, wherein code for providing the training set comprises code for selecting data points for stimuli having a level of activity sufficient to induce a change greater than a defined magnitude in at least some phenotypic features.

88. The computer program product of claim 87, wherein selecting data points is performed for stimuli indicated to exhibit hepatotoxicity.

89. The computer program product of claim 87, wherein selecting data points is performed for stimuli indicated to exhibit hepatotoxicity and for stimuli not indicated to exhibit hepatotoxicity.

90. A computer program product comprising a machine readable medium on which is provided program instructions for classifying a stimulus according to whether it is hepatotoxic, the instructions comprising:
code for receiving at least one image of hepatocytes which have been exposed to the stimulus and treated with one or more markers for cellular components in the hepatocytes;
code for automatically extracting two or more phenotypic features from the one or more markers in the image, wherein at least one of the features is extracted from segmented regions within the hepatocyte images, which segmented regions correspond to granules and/or peripheral regions within the hepatocytes;
code for applying the two or more phenotypic features to a model for classifying stimuli according to whether they are hepatotoxic; and
code for receiving a hepatatotoxicity classification for the stimulus as an output from the model.

91. The computer program product of claim 90, wherein at least one of the one or more markers comprises a marker for a cytoskeletal protein or structure, a marker for a canalicular component, a marker for an endocytic component, a marker for a mitochondrial component, a marker for nuclear component, a marker for a Golgi component, a marker for general protein content within a cell, or a marker for a lipid.

92. The computer program product of claim 90, wherein the markers from which the phenotypic features are extracted comprise at least one marker for a neutral lipid and at least one marker for a phospholipid.

93. The computer program product of claim 90, wherein the markers from which the phenotypic features are extracted comprise at least one marker for BSEP and at least one marker for MRP2.

94. The computer program product of claim 90, wherein at least one of the features is extracted from segmented regions corresponding to nuclei within the hepatocytes.

95. The computer program product of claim 90, wherein at least one of the features is extracted from segmented regions corresponding to whole cells.

96. The computer program product of claim 90, wherein the model for classifying stimuli comprises a decision tree.

97. The computer program product of claim 90, further comprising code for, prior to applying the two or more phenotypic features to a model, determining that the stimulus has a level of activity sufficient to induce a change greater than a defined magnitude in at least some phenotypic features.

98. The computer program product of claim 90, wherein the program instructions comprise code for classifying a stimulus according to whether it is hepatotoxic without determining whether the stimulus has a level of activity sufficient to induce a change greater than a defined magnitude in at least some phenotypic features.
